(19) United States
(12) Patent Application Publication
(10) Pub. No.: US 2005/0234025 A1
(43) Pub. Date: Oct. 20, 2005

(54) Compositions comprising one or more policosanols and/or policosanoic acids combined with sterol and/or steroid based ascorbic acid derivatives, and uses thereof

(75) Inventors: James P. Kutney, Vancouver (CA); Laura Marie Wessman, Vancouver (CA)

Correspondence Address: SUSAN M. BEN-OLIEL;BARRISTER & SOLICITOR/PATENT AGENT, 2983 West 41st Avenue, VANCOUVER, BC, V6N 3C8, CA

(73) Assignee: FORBES MEDI-TECH INC.

(21) Appl. No.: 10/827,537

(22) Filed: Apr. 20, 2004

(51) Int. Cl. A61K 31/56; A61K 31/375; A61K 31/045; A61K 31/20

(52) U.S. Cl. 514/171; 514/474; 514/558; 514/724

(57) ABSTRACT

A composition comprises one or more long chain alcohols (policosanols) and/or their respective acids (policosanoic acids) and one or more ascorbic acid derivatives, including all biologically acceptable salts or solvates or prodrugs of at least one such derivative or of the salts or of the solvates thereof and is used for the treatment and prevention of a variety of conditions and disorders.

### FIELD OF THE INVENTION

This present invention relates to the field of compositions comprising specific naturally derived compounds.

### BACKGROUND OF THE INVENTION

While recent advances in science and technology are helping to improve quality and add years to human life, the prevention of atherosclerosis, the underlying cause of cardiovascular disease (“CVD”) has not been sufficiently addressed. Atherosclerosis is a degenerative process resulting from an interplay of inherited (genetic) factors and environmental factors such as diet and lifestyle. Research to date suggest that cholesterol may play a role in atherosclerosis by forming atherosclerotic plaques in blood vessels, ultimately cutting off blood supply to the heart muscle or alternatively to the brain or limbs, depending on the location of the plaque in the arterial tree^{1,2}. Data from the early Framingham Epidemiological Study indicates that increases in serum cholesterol levels are associated with increased risk of death from CVD³. More recent studies confirm that CVD is a leading cause of death and disability in industrialized nations⁴.

Studies have indicated that a 1% reduction in a person's total serum cholesterol yields a 2% reduction in risk of a coronary artery event⁵. Statistically, a 10% decrease in average serum cholesterol (e.g. from 6.0 mmol/L to 5.3 mmol/L) may result in the prevention of 100,000 deaths in the United States annually⁶.

As a result, experts have recommended a program for those with or at high risk of presenting CVD to take measures to lower serum low density lipoprotein cholesterol (“LDL-C”) and total cholesterol (“TC”) through a combination of measures including dietary changes, physical exercise, and lifestyle adjustment. Often, and despite these measures, for many individuals, intervention by way of pharmaceutical agents is still required in order to control CVD risk factors. The most potent drugs that are currently used to lower elevated LDL-C are the 3-hydroxy-3-methylglutaryl-coenzyme A (HMG-CoA) reductase inhibitors, also known as statins. The anti-cholesterolemic activity of lovastatin is due to the inhibition of HMG-CoA reductase which catalyses a major rate-limiting step in the biosynthesis of cholesterol in the mevalonate pathway. The inhibition is a result of the structural similarity of the acid form of the statin to the natural substrate of the enzyme (HMG-CoA).

Unfortunately, this same biosynthetic pathway is also shared by Co-enzyme Q10. Therefore, one unfortunate consequence of statin drugs is the unintentional inhibition of CoQ10 synthesis. Thus, in the long run, statin drugs could predispose the patients to heart disease by lowering their CoQ10 status, the very condition that these drugs are intended to prevent. Further, the reduction of CoQ10 levels might be associated with myopathy, an adverse effect associated with statin drugs. This metabolic myopathy is related to ubiquinone (CoQ10) deficiency in muscle cell mitochondria, disturbing normal cellular respiration and causing adverse effects such as rhabdomyolysis, exercise intolerance, and recurrent myoglobinuria.^{7a}

As CVD patients become increasingly wary of these drug therapies, and potential side and long terms adverse effects, the need for naturally derived, safe and effective agents which address the underlying causes of CVD, becomes even more apparent.

One focus of such alternative research has been on policosanols. Policosanol was a name originally given to a unique extract of Cuban sugarcane (Saccharum officinarum L), the origins of which read more like a socio-political treatise than a scientific endeavour. In 1964, Che Guevara, Cuba's Minister of Industry, created the revolution's first state-sponsored research centre, the Cuban Institute for Research on Sugar Cane Derivatives.^{7b} The purpose of this institute was to create sugarcane derivates potentially to surpass the value of refined white sugar. The first initiative, policosanol derived from Cuban sugar cane, has been found to comprise eight long chain alcohols in certain relative amounts, and is the subject of a number of patents including U.S. Pat. No. 5,663,156 and U.S. Pat. No. 5,856,316, both to Granja et al. Dalmer Laboratories in Cuba continues the production of and research into policosanols.

In general though, policosanols are mixtures of high molecular weight aliphatic alcohols derived from waxy materials, plants (commonly though from sugar cane wax⁸) which have been shown to lower elevated serum cholesterol levels in addition to exhibiting anti-thrombotic effects.⁹⁻¹³ Animal studies over the last decade, including human studies, have shown that policosanols are effective anti-platelet agents^{14,15-18}, although the precise dosage regimen remains unclear. Recent studies have shown that policosanols lower cholesterol levels by reducing cholesterol biosynthesis, while enhancing LDL cholesterol receptor mediated clearance. One group administered policosanols orally at a dosage of 50 mg/kg/day to casein-induced hypercholesterolaemic rabbits for 30 days.¹⁹ It was observed that the incorporation of 3H-water into the hepatic sterols was significantly depressed while clearance of LDL cholesterol was significantly improved. This suggests that policosanols inhibit cholesterol biosynthesis and enhance LDL decatabolism. Further research suggests that policosanols possibly suppress the action of HMG-CoA reductase, as per the statin family of drugs.²⁰

Another focus of such research related to naturally derived, safe and effective agents to address the underlying causes of CVD has been plant-derived sterols. Sterols are naturally occurring compounds that perform many critical cellular functions. Phytosterols such as campesterol, stigmasterol and beta-sitosterol in plants, ergosterol in fungi and cholesterol in animals are each primary components of cellular and sub-cellular membranes in their respective cell types. The dietary source of phytosterols in humans comes from plant materials i.e. vegetables and plant oils. The estimated daily phytosterol content in the conventional western-type diet is approximately 60-80 milligrams in contrast to a vegetarian diet which would provide about 500 milligrams per day.

Phytosterols have received a great deal of attention due to their ability to decrease serum cholesterol levels when fed to a number of mammalian species, including humans. While the precise mechanism of action remains largely unknown, the relationship between cholesterol and phytosterols is apparently due in part to the similarities between the respective chemical structures (the differences occurring in the side chains of the molecules). It is assumed that phytosterols displace cholesterol from the micellar phase and thereby reduce its absorption or possibly compete with receptor and/or carrier sites in the cholesterol absorption process.

Over forty years ago, Eli Lilly marketed a sterol preparation from tall oil and later from soybean oil called Cytellin™ which was found to lower serum cholesterol by about 9% according to one report. Various subsequent researchers have explored the effects of sitosterol preparations on plasma lipid and lipoprotein concentrations²² and the effects of sitosterol and campesterol from soybean and tall oil sources on serum cholesterols.²³ Compositions have been explored in which phytosterols or phytostanols (their hydrogenated counterparts) are esterified in order to enhance solubility. One composition of phytosterols which has been found to be highly effective in lowering serum cholesterol is disclosed in U.S. Pat. No. 5,770,749 to Kutney et al.

It is known to combine phytosterols and policosanols to lower serum cholesterol: U.S. Pat. No. 5,952,393, U.S. Pat. No. 6,197,832, U.S. Patent Application 20010034338, all to Sorkin; and New Zealand Patent 508692 to McNeil PPC Inc. U.S. Patent Application 20020183298 to Schersl describes the use of “wood derived” long chain alcohols and the combination of such alcohols with phytosterols, whether free or esterified with simple carboxylic acids. U.S. Patent Application 20020016314 also to Schersl describes the use of simple policosanol ethyl or methyl esters and combination with phytosterols (free or esterified).

Another potential focus of alternative CVD research, particularly in relation to anti-inflammatory indications relates to steroids, specifically the downstream metabolites of dehydroepiandrostrone (DHEA), particularly androstenediol (5-androstene-3β,17β-diol or AED) and androstenetriol (5-androstene-3β,7β, 17β-triol or AET).

U.S. Pat. No. 5,804,575 to Schwartz et al. teaches DHEA derivatives and their use as anti-cancer, anti-obesity, anti-diabetic and hypolipidemic agents. Related DHEA-derivative patents to Schwartz include U.S. Pat. Nos.: 4,898,694; 5,001,119; 5,028,631; 5,157,031; 5,700,793; 5,714,481; and 5,744,462. Ben-David, et al.²⁴ observed that DHEA treatment has an anti-hypercholesterolemic effect in mice.

Overall, DHEA and its metabolites are considered to be potent agents useful in a number of conditions and disorders, particularly as immunomodulating and anti-inflammatory compounds. More recently, the role of inflammation in CVD is becoming more understood. For example, Ricker et al.²⁵ describes a possible role of inflammation in the CVD process. J. Boyle²⁶ suggests an association between plaque rupture and atherosclerotic inflammation.

Despite the obvious and now well recorded advantages of phytosterols, not only in the treatment of CVD and its underlying conditions such as hypercholesterolemia, hyperlipidemia, atherosclerosis, hypertension, thrombosis but in the treatment of other diseases such as Type II diabetes, dementia cancer and aging, the administration of phytosterols and the incorporation thereof into foods, pharmaceuticals and other delivery vehicles has been complicated by the fact that they are highly hydrophobic (i.e. they have poor water solubility). Studies have investigated how the form (for example crystalline, suspension, granular) in which the phytosterols are dosed impacts on their ability to lower serum cholesterol levels. As phytosterols are highly hydrophobic, they do not dissolve to any appreciable extent in the micellar phase in the digestive tract and therefore are not capable of efficiently blocking cholesterol absorption. Oils and fats are capable to a limited but not satisfactory degree of dissolving free phytosterols. Only solubilized phytosterols inhibit the absorption of cholesterol.

A similar, significant obstacle to the efficient use of the androstene and androstane family of compounds in the treatment or prevention of various disorders is their poor solubility.

Accordingly, despite various drawbacks, research has focussed, to some extent, on natural, safe alternatives to drugs such as statins, with the goal being the finding of compounds or compositions which equal or better the efficacy of such drugs, which are readily amenable to incorporation into various delivery vehicles and which can be administered in a state which optimizes their therapeutic availability.

It is an object of the present invention to obviate or mitigate the above noted disadvantages.

### SUMMARY OF THE INVENTION

The present invention provides a composition comprising one or more long chain alcohols (policosanols) and/or their respective acids (policosanoic acids) and one or more ascorbic acid derivatives, including all biologically acceptable salts or solvates or prodrugs of at least one such derivative or of the salts or of the solvates thereof represented by the following general formulae: 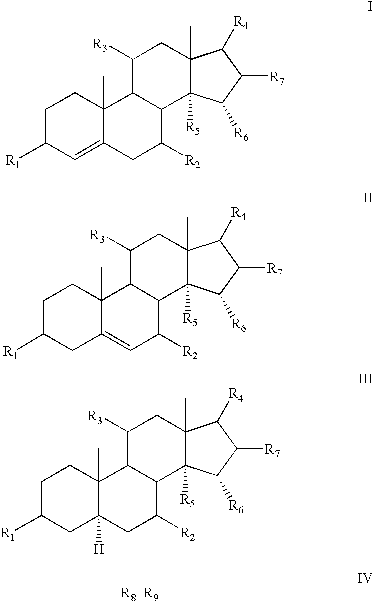
wherein, in formulae I, II and III, representing derivatives of androstene and androstane, R₁, R₂, R₃, R₄, R₅, R₆ may individually be chosen from hydrogen, OH, carbonyl, and an ascorbyl-based moiety, and wherein at least one substituent is the ascorbyl-based moiety; and R₇ may be hydrogen or any halogen; and wherein, in formula IV, R8 is an ascorbyl-based moiety and R9 is a sterol or stanol moiety.

The present invention further comprises compositions for treating and/or preventing a plurality of diseases, conditions and disorders including, but not limited to, treating and/or preventing CVD and its underlying manifestations including atherosclerosis, hypercholesterolemia, hyperlipidemia, hypertension, thrombosis, coronary artery disease, aneurysm, myocardial infarction, embolism, stroke, thrombosis, angina or unstable angina, coronary plaque inflammation, related diseases such as Type II diabetes, as well as treating diseases, conditions or disorders in which immune function is compromised or in which immune system enhancement is required, including radiation-related injuries, HIV, AIDS, hepatitis, chronic fatigue syndrome, and malaria, as well as reducing inflammation, caused by, for example bacterial-induced inflammation, viral-induced inflammation, chronic inflammatory bowel disease and inflammation associated with surgical procedures and injury, as well as being useful to control weight gain or promote weight loss, as well as being useful in preventing cancer, as well as exhibiting anti-aging effects which comprise one or more long chain alcohols (policosanols) and/or their respective acids (policosanoic acids) and one or more ascorbic acid derivatives, having one or more of formulae I, II, III, and a pharmaceutically acceptable or non-toxic food quality carrier therefor.

The present invention further comprises compositions for treating and/or preventing a plurality of diseases, conditions and disorders including, but not limited to, treating and/or preventing CVD and its underlying manifestations including atherosclerosis, hypercholesterolemia, hyperlipidemia, hypertension, thrombosis, coronary artery disease, coronary plaque inflammation, as well as exhibiting anti-oxidant effects which comprise one or more long chain alcohols (policosanols) and/or their respective acids (policosanoic acids) and one or more ascorbic acid derivatives, having formulae IV, and a pharmaceutically acceptable or non-toxic food quality carrier therefor.

The present invention further provides a method for treating and/or preventing a plurality of diseases, conditions and disorders including, but not limited to, treating and/or preventing CVD and its underlying manifestations including atherosclerosis, hypercholesterolemia, hyperlipidemia, hypertension, thrombosis, coronary artery disease, aneurysm, myocardial infarction, embolism, stroke, thrombosis, angina or unstable angina, coronary plaque inflammation, related diseases such as Type II diabetes, as well as treating diseases, conditions or disorders in which immune function is compromised or in which immune system enhancement is required, including radiation-related injuries, HIV, AIDS, hepatitis, chronic fatigue syndrome, and malaria, as well as reducing inflammation, caused by, for example bacterial-induced inflammation, viral-induced inflammation, chronic inflammatory bowel disease and inflammation associated with surgical procedures and injury, as well as being useful to control weight gain or promote weight loss, as well as being useful in preventing cancer, as well as exhibiting anti-aging effects by administering to an animal, particularly a human, a composition comprising one or more long chain alcohols (policosanols) and/or their respective acids (policosanoic acids) and one or more ascorbic acid derivatives, having one or more of the formulae I, II and III or III.

The present invention further provides a method for treating and/or preventing a plurality of diseases, conditions and disorders including, but not limited to, treating and/or preventing CVD and its underlying manifestations including atherosclerosis, hypercholesterolemia, hyperlipidemia, hypertension, thrombosis, coronary artery disease, coronary plaque inflammation, as well as exhibiting anti-oxidant effects by administering to an animal, particularly a human, a composition comprising one or more long chain alcohols (policosanols) and/or their respective acids (policosanoic acids) and one or more ascorbic acid derivatives, having the formula IV.

The compositions of the present invention, comprising select natural compounds, have numerous advantages over drugs which are known and described in the art. For example, with respect to lowering serum LDL cholesterol, there is a complementary or greater than additive effect by administration of the composition in accordance with the present invention as compared to administration of the individual components. Further, these particular compositions have numerous additional advantages. In particular, solubility of the ascorbyl-based constituent in aqueous solutions such as water is improved thereby allowing oral administration per se. Likewise, other modes of administration are facilitated. These compositions can be prepared and used as such or they can be easily incorporated into pharmaceutical preparations, optionally in conjunction with other therapeutic agents.

This generally translates into lower administration dosages of the constituents of the composition in order to achieve the desired therapeutic effect.

These effects and other significant advantages are described in more detail below.

### BRIEF DESCRIPTION OF THE DRAWINGS

The present invention is illustrated by way the following non-limiting drawings in which:

FIG. 1 is a schematic showing the synthesis of one preferred derivative of the present invention, disodium ascorbyl phosphate ester of dehydroisoandrosterone;

FIG. 2 is a schematic showing the synthesis of one preferred derivative of the present invention, disodium ascorbyl phosphate ester of 5α-Androstan-3β-ol-17-one;

FIG. 3 is a schematic showing the synthesis of one preferred derivative of the present invention, disodium ascorbyl phosphate ester of Androst-5-ene-3β,17β-diol;

FIG. 4 is a schematic showing the synthesis of one preferred derivative of the present invention, disodium ascorbyl phosphate ester of Androst-5-ene-17β-ol;

FIG. 5 is a schematic showing the synthesis of one preferred derivative of the present invention, tetra-sodium monoascorbyl diphosphate ester of 3β-acetoxyandrost-5-ene-7β,17β-diol;

FIG. 6 is a schematic showing the synthesis of one preferred derivative of the present invention, tetrasodium diascorbyl diphosphate ester of Androst-5-ene-3β,17β-diol;

FIG. 7 is a schematic showing a process of preparing phytostanol-phosphate-ascorbate and its sodium salt;

FIG. 8 is a schematic showing a process of preparing phytostanol-carbonate-ascorbate and its sodium salt; and

FIG. 9 is a schematic showing a process of preparing phytostanol-oxalate-ascorbate and its sodium salt.

### PREFERRED EMBODIMENTS OF THE INVENTION

The following detailed description is provided to aid those skilled in the art in practising the invention. However this detailed description should not be construed so as to unduly limit the scope of the present invention. Modifications and variations to the embodiments discussed herein may be made by those with ordinary skill in the art without departing from the spirit or scope of the present invention.

As used herein, “animal” means any member of the animal kingdom, including all mammals and most preferably humans. Veterinary use is also contemplated.

As used herein, the term “compound” is interchangeable with the terms “derivative”, “structure” and “analogue”.

As used herein, the term “prodrug” refers to compounds that are drug precursors, which, following administration to a patient, release the drug in vivo via some chemical or physiological process (for example, a prodrug, on being brought to physiological pH or through enzyme action is converted to the desired drug form).

As used herein, the term “solvate” refers to a molecular or ionic complex of molecules or ions of solvent with those of solute (for example the compounds of formulae a) to 0 or prodrugs of compounds a) to f)). Non-limiting examples of useful solvents include polar, protic solvents such as water and/or alcohols (for example, methanol).

As used herein, the terms “effective” or “therapeutically effective”, are intended to qualify the amount of the compound(s) or composition administered to an animal, in particular a human, in order to elicit a biological or medical response of a cell, tissue, system, animal or mammal that is being sought by the person administering the compound(s) or composition and which amount achieves one or more of the desired treatment and/or preventative goals. With respect to the compositions of the present invention, comprising compounds of formulae I, II or III, these goals include:
1) decreasing serum LDL cholesterol;
2) increasing serum HDL cholesterol;
3) decreasing serum triglycerides;
4) preventing, reducing, eliminating or ameliorating a dyslipidemic condition or disorder;
5) preventing, reducing, eliminating or ameliorating hypercholesterolemia or hypoalphalipoproteinemia;
6) preventing, reducing, eliminating or ameliorating the development of atherosclerotic lesions or plaque formation;
7) modulating an immune response;
8) reducing inflammation;
9) modifying viral, bacterial or parasitic activity;
10) stimulating myelopoiesis;
11) enhancing resistance to bacterial, parasitic and/or viral infection;
12) providing protection from radiation or to restore immunity after a radiation injury;
13) controlling weight gain or promoting weight loss;
14) treating or managing the symptoms of diabetes; and
15) treating cancer.

With respect to the compositions of the present invention, comprising compounds of formula IV, these goals include:
1) decreasing serum LDL levels;
2) increasing serum HDL levels;
3) decreasing serum triglyceride levels;
4) preventing, reducing, eliminating or ameliorating a dyslipidemic condition or disorder;
5) preventing, reducing, eliminating or ameliorating hypercholesterolemia or hypoalphalipoproteinemia;
6) preventing, reducing, eliminating or ameliorating the development of atherosclerotic lesions or plaque formation;
7) reducing coronary plaque inflammation;
8) exhibiting anti-oxidant effects; and
9) preventing, reducing, eliminating or ameliorating any condition, disease or disorder which has as its basis or which is exacerbated by a deficiency in plasma HDL, or by an excess of either LDL, VLDL, Lp(a), beta-VLDL, IDL or remnant lipoproteins.

As used herein, the term “policosanols” refers to all saturated and unsaturated long chain alcohols, specifically higher primary aliphatic alcohols, which can be isolated from a variety of natural sources, including, but not limited to, sugar cane wax, beeswax, and rice bran wax. The most preferred source is sugar cane wax.

Policosanols occur naturally in this wax form and are characterized by fatty alcohol chains ranging from 20 to 39 carbon atoms in length. Accordingly, the chemical structure for policosanols is CH₃—(CH₂)ₙ—CH₂OH, wherein n is from 18 to 37. Isolated mixtures of policosanols primarily comprise octacosanol, triacontanol, and hexacosanol. It is to be understood that policosanols, as used herein, encompasses all long chain alcohols, regardless of source or other nomenclature, and includes, for example, apisols as derived from beeswax (solvent extraction process described in U.S. Pat. No. 6,225,354 to Perez, incorporated herein by reference.)

As used herein, the term “policosanoic acids” refers to the respective long chain carboxylic acids of the long chain alcohols defined above. These long chain acids are preferably isolated from the same sources as policosanols.

As used herein, the term “sterol” includes all sterols without limitation, for example: (from any source and in any form: α, β and γ) sitosterol, campesterol, stigmasterol, brassicasterol (including dihydrobrassicasterol), desmosterol, chalinosterol, poriferasterol, clionasterol, ergosterol, coprosterol, codisterol, isofucosterol, fucosterol, clerosterol, nervisterol, lathosterol, stellasterol, spinasterol, chondrillasterol, peposterol, avenasterol, isoavenasterol, fecosterol, pollinastasterol, cholesterol and all natural or synthesized forms and derivatives thereof, including isomers.

The term “stanol” refers to, for example: (from any source and in any form: α, β and γ) saturated or hydrogenated sterols including all natural or synthesized forms and derivatives thereof, and isomers, including sitostanol, campestanol, stigmastanol, brassicastanol (including dihydrobrassicastanol), desmostanol, chalinostanol, poriferastanol, clionastanol, ergostanol, coprostanol, codistanol, isofucostanol, fucostanol, clerostanol, nervistanol, lathostanol, stellastanol, spinastanol, chondrillastanol, pepostanol, avenastanol, isoavenastanol, fecostanol, and pollinastastanol.

It is to be understood that modifications to the sterols and stanols i.e. to include side chains also falls within the purview of this invention. It is also to be understood that, when in doubt throughout the specification, and unless otherwise specified, the term “sterol” encompasses both sterol and stanol. The terms “phytosterol” and “phytostanol” may also be used and refer to all p/ant-derived sterols or stanols respectively.

The sterols and stanols for use in forming derivatives in accordance with this invention may be procured from a variety of natural sources or they may be artificially synthesized. For example, they may be obtained from the processing of plant oils (including aquatic plants) such as corn oil and other vegetable oils, wheat germ oil, soy extract, rice extract, rice bran, rapeseed oil, sunflower oil, sesame oil and fish (and other marine-source) oils. They may also be derived from yeasts and fungi, for example ergosterol. Accordingly, the present invention is not to be limited to any one source of sterols. U.S. Pat. No. 4,420,427 teaches the preparation of sterols from vegetable oil sludge using solvents such as methanol. Alternatively, phytosterols and phytostanols may be obtained from tall oil pitch or soap, by-products of forestry practises as described in U.S. Pat. No. 5,770,749, incorporated herein by reference. A further method of extracting sterols and stanols from tall oil pitch is described in Canadian Patent Application Serial No. 2,230,373 which was filed on Feb. 20, 1998 (corresponding to PCT/CA99/00150 which was filed on Feb. 19, 1999) and U.S. patent application Ser. No. 10/060,022 which was filed on Jan. 28, 2002 the contents of all of which are incorporated herein by reference.

Accordingly, it is to be understood that the widest possible definition is to be accorded to the terms “sterol” and “stanol” as used herein. Thus, the terms “sterols” and “stanols” encompass all analogues, which may further have a double bond at the 5-position in the cyclic unit as in most natural sterols, or one or more double bonds at other positions in the rings (for example, 6, 7, 8(9), 8(14), 14 5/7) or no double bonds in the cyclic unit as in stanols. Further, there may be additional methyl groups as, for example, in \acute{α}₁-sitosterol.

Sterols are naturally occurring compounds that perform many critical cellular functions. Sterols such as campesterol, stigmasterol and beta-sitosterol in plants, ergosterol in fungi and cholesterol in animals are each primary components of cellular and sub-cellular membranes in their respective cell types. Phytosterols, in particular, have received a great deal of attention due to their ability to decrease serum cholesterol levels when fed to a number of mammalian species, including humans.

According to one aspect of the present invention, there are provided novel compositions suitable for use per se in treating or preventing a wide variety of diseases, conditions and disorders and which comprise one or more long chain alcohols (policosanols) and/or their respective acids (policosanoic acids) and one or more ascorbic acid derivatives, including all biologically acceptable salts or solvates or prodrugs of at least one such derivative or of the salts or of the solvates thereof represented by the following general formulae: 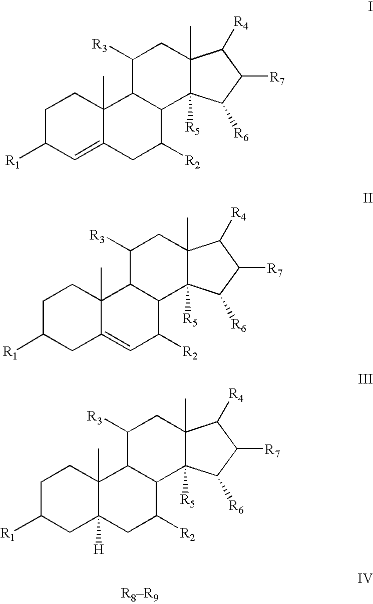
wherein, in formulae I, II and III, representing derivatives of androstene and androstane, R₁, R₂, R₃, R₄, R₅, R₆ may individually be chosen from hydrogen, OH, carbonyl, and an ascorbyl-based moiety, and wherein at least one substituent is the ascorbyl-based moiety; and R₇ may be hydrogen or any halogen; and wherein, in formula IV, R8 is an ascorbyl-based moiety and R9 is a sterol or stanol moiety.

The components of this composition are described in more detail herein. It should be noted that, throughout this disclosure, the terms “compound”, “derivative”, “structure” and “analogue” are used interchangeably to describe the novel unitary compound which links or couples a “sterol” or “steroid” moiety to an ascorbyl-based moiety. This compound is then admixed with one or more policosanols and/or their respective acids (policosanoic acids) in order to form the composition of the present invention.

Policosanol, as used in the composition of the present invention, may be one or a mixture of more than one long chain alcohols. Generally, if a mixture of policosanols is used, it comprises greater than 90% by weight of alcohols which are C₂₀ or longer. Preferably, if a mixture of policosanols is used, it comprises greater than 50% by weight of octacosanol (C₂₈), more preferably greater then 60% by weight octacosanol. Policosanol components and relative concentrations are described in U.S. Pat. No. 5,856,316, the contents of which are incorporated herein by reference.

The preferred source of policosanols is sugar cane wax, most preferably Cuban sugar cane wax. Nonetheless, other sources, including beeswax and rice bran wax may be used, although it should be recognized that the concentration of octacosanol is lower in mixtures from these sources. In addition, long chain alcohols can also be synthesized de novo using techniques well known in the art.

Extraction processes are described in U.S. Patent Application 20040034241, U.S. Pat. No. 5,856,316 and New Zealand Patent 508,692. Methods of preparation are also described in WO 03/106397.

One preferred policosanol mixture, derived from purified sugar cane wax, comprises octacosanol, triacontanol, hexacosanol and other C₂₂-C₃₄ alcohols in more minor quantities. It is preferred that octacosanol comprise at least 50% by weight of the mixture. A preferred mixture comprises at least 60% by weight octacosanol, from 9-15% triacontanol and from 4-10% hexacosanol. One ideal formulation is extracted from Cuban sugar cane wax and comprises octacosanol (C₂₈, approx. 62.9%), triacontanol (C₃₀, approx 12.6%), hexacosanol (C₂₆, approx 6.2%) and other C₂₂-C₃₄ alcohols in more minor quantities. An example of a commercially available form of this mixture is sold under the trade mark Ateromixol™.

The present invention also provides that the composition may comprise one or more acids of policosanols. Preferably, if a mixture of policosanoic acids are used, it comprises greater than 50% by weight of octacosanoic acid (C₂₈), more preferably greater then 60% by weight octacosanoic acid. The preferred mixture of acids may also comprise dotriacontanoic acid (C₃₂) and tetracontanoic acid (C₃₄). Such mixtures of long chain carboxylic acids have been shown as useful cholesterol lowering agents in rabbits²¹ and more recent human studies have confirmed this LDL cholesterol lowering effect.²² These acids may be extracted from the source material by methods described and referenced in the literature ^{.22}

Notwithstanding the above, it is considered fully within the scope of the present invention to use any one or more of the policosanols and/or policosanoic acids isolated or synthesized de novo. For example, octacosanol and/or octacosanoic acid may be combined with one or more of the compounds of formulae I-IV to form a composition.

For many years, it has been recognized that L-ascorbic acid (commonly known as vitamin C) is a vital part of balanced human nutrition and plays a role as a physiological anti-oxidant. However, ascorbic acid is the least stable vitamin with which to work since it reacts extremely easily with atmospheric oxygen yielding dehydroascorbic acid which further and readily decomposes into compounds void of vitamin C efficacy. The composition of the present invention takes advantage of ascorbyl analogues in which the ascorbic acid is protected from such decomposition. Furthermore, it is believed that the therapeutic effects of each component of the composition of the present invention are enhanced in a synergistic or additive fashion by virtue of association with the other components. These advantages have not heretofore been appreciated or explored.

In a most preferred form of the present invention, the ascorbyl moiety which is coupled to the sterol or steroid moiety is selected individually from one or more of the following structures: 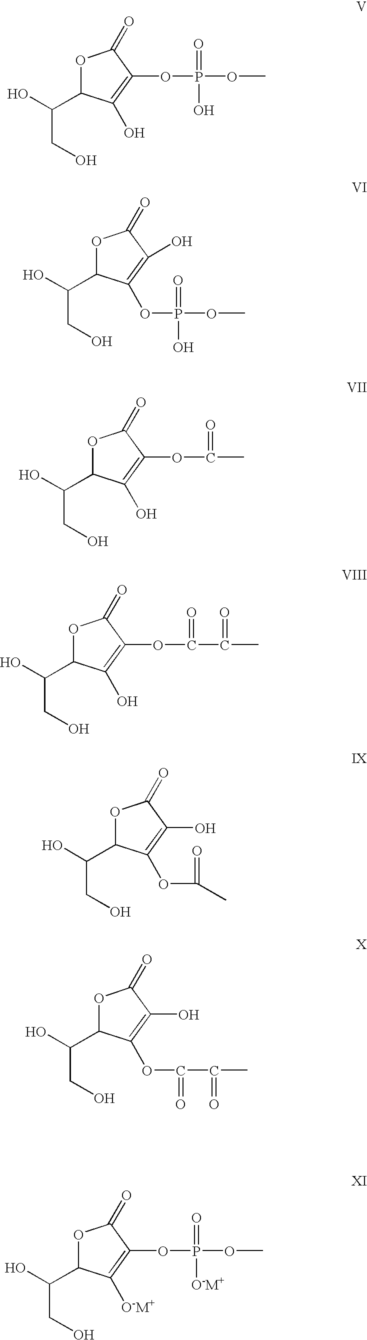 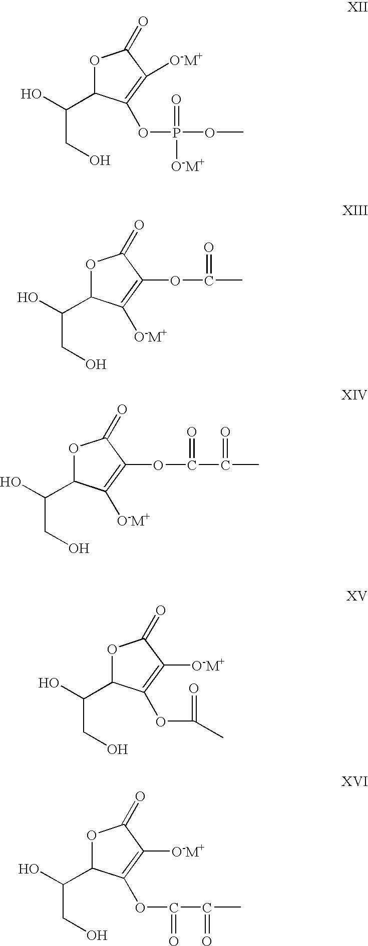
wherein M+is selected independently, from the group consisting of hydrogen, any metal, alkali earth metal, or alkali metal. Preferably, M+ is selected from the group consisting of calcium, magnesium, manganese, copper, zinc, sodium, potassium and lithium.
Compositions Comprising Policosanols and/or Their Respective Acids (Policosanoic Acids) and Compounds of Formulae I, II and III:

In one aspect, the compositions of the present invention comprise one or more long chain alcohols (policosanols) and/or their respective acids (policosanoic acids) and compounds represented by one or more of formulae I, II, and III noted above and wherein the substituents R1-R7 are selected from one or more of the following combinations:
1) wherein R1 is an ascorbyl moiety, R2, R3, R5, R6 and R7 are H, and R4 is carbonyl;
2) wherein R1 is an ascorbyl moiety, R2, R3, R5 R6 and R7 are H, and R4 is OH;
3) wherein R4 is an ascorbyl moiety, R1 is OH, and R2, R3, R5, R6 and R7 are H;
4) wherein R4 is an ascorbyl moiety, R1 is carbonyl, and R2, R3, R5, R6 and R7 are H;
5) wherein R1 and R4 are ascorbyl moieties, and R2, R3, R5, R6, and R7 are H;
6) wherein R1 and R2 are ascorbyl moieties, R3, R5, R6 and R7 are H, and R4 is OH;
7) wherein R1 and R2 are ascorbyl moieties, R3, R5, R6, and R7 are H, and R4 is carbonyl;
8) wherein R1 and R4 are ascorbyl moieties, R2 is OH, and R3, R5, R6 and R7 are H;
9) wherein R3 is an ascorbyl moiety, R1 and R4 are carbonyl, and R2, R5, R6 and R7 are H;
10) wherein R3 is an ascorbyl moiety, R1 and R4 are OH, and R2, R5, R6 and R7 are H;
11) wherein R5 is an ascorbyl moiety, R1 and R4 are carbonyl, and R2, R3, R6 and R7 are H;
12) wherein R5 is an ascorbyl moiety, R1 and R4 are OH, and R2, R3, R6 and R7 are H;
13) wherein R6 is an ascorbyl moiety, R1 and R4 are carbonyl, and R2, R3, R5 and R7 are H;
14) wherein R6 is an ascorbyl moiety, R1 and R4 are OH, and R2, R3, R5 and R7 are H;
15) wherein R4 is an ascorbyl moiety, R1 and R2 are OH, and R3, R5, R6 and R7 are H;
16) wherein R4 is an ascorbyl moiety, R1 and R3 are OH, and R2, R5, R6 and R7 are H;
17) wherein R4 is an ascorbyl moiety, R1 and R3 are carbonyl, and R2, R5, R6 and R7 are H;
18) wherein R4 is an ascorbyl moiety, R1 is OH, R3 is carbonyl and R2, R5, R6 and R7 are H;
19) wherein R4 is an ascorbyl moiety, R1 is carbonyl, R3 is OH, and R2, R5, R6 and R7 are H;
20) wherein R1 is an ascorbyl moiety, R3 and R4 are OH, and R2, R5, R6 and R7 are H;
21) wherein R1 is an ascorbyl moiety, R3 and R4 are carbonyl, and R2, R5, R6 and R7 are H;
22) wherein R1 is an ascorbyl moiety, R3 is OH, R4 is carbonyl, and R2, R5, R6 and R7 are H;
23) wherein R1 is an ascorbyl moiety, R3 is carbonyl, R4 is OH, and R2, R5, R6 and R7 are H;
24) wherein R1 is an ascorbyl moiety, R2 and R4 are OH, and R3, R5, R6 and R7 are H;
25) wherein R1, R2 and R4 are ascorbyl moieties, and R3, R5, R6 and R7 are H;
26) wherein R1 and R2 are ascorbyl moieties, R4 is carbonyl, and R3, R5, R6 and R7 are H;
27) wherein R1 is an ascorbyl moiety, R4 is carbonyl, R2, R3, R5, R6 are H, and R7 is a halogen;
28) wherein R1 and R4 are ascorbyl moieties, R2, R3, R5, R6 are H, and R7 is a halogen;
29) wherein R4 is an ascorbyl moiety, R1 is carbonyl, R2, R3, R5, R6 are H, and R7 is a halogen;
30) wherein R3 is an ascorbyl moiety, R4 is carbonyl, R1 is OH, R2, R5, R6 are H, and R7 is a halogen;
31) wherein R3 is an ascorbyl moiety, R4 is OH, R1 is carbonyl, R2, R5, R6 are H, and R7 is a halogen;
32) wherein R5 is an ascorbyl moiety, R1 and R4 are carbonyl, R2, R3, R6 are H, and R7 is a halogen;
33) wherein R5 is an ascorbyl moiety, R1 and R4 are OH, R2, R3, R6 are H, and R7 is a halogen;
34) wherein R6 is an ascorbyl moiety, R1 and R4 are carbonyl, R2, R3, R5 are H, and R7 is a halogen;
35) wherein R6 is an ascorbyl moiety, R1 and R4 are OH, R2, R3, R5 are H, and R7 is a halogen;
36) wherein R1, R3 and R4 are ascorbyl moieties, R2 and R5, R6 are H, and R7 is halogen;
37) wherein R1, R4 and R5 are ascorbyl moieties, R2 and R3, R6 are H, and R7 is halogen;
38) wherein R1 R2 and R4 are ascorbyl moieties, R3, R5, and R6 are H, and R7 is a halogen; and
39) wherein R1, R4, R6 are ascorbyl moieties; R2, R3, and R5 are H; and R7 is a halogen.

It is to be understood that these preferred derivatives include all biologically acceptable salts thereof. Halogens include chlorine (Cl), bromine (Br), fluorine (F) and iodine (1). Preferably, the ascorbyl-based moiety is as defined in formulae V to XVI.

There are many processes by which structures joining compounds within the androstane and androstene family and ascorbic acid can be formed. Exemplary methods are described in PCT/CA03/00824, the contents of which are incorporated herein by reference.

In general, the selected steroid (or halophosphate, halocarbonate or halo-oxalate derivatives thereof) and ascorbic acid are mixed together under reaction conditions to permit condensation of the “acid” moiety with the “alcohol” (steroid). These conditions are the same as those used in other common esterification reactions such as the Fisher esterification process in which the acid component and the alcohol component are allowed to react directly or in the presence of a suitable acid catalyst such as mineral acid, sulfuric acid, phosphoric acid, p-toluenesulfonic acid. The organic solvents generally employed in such esterification reactions are ethers such as diethyl ether, tetrahydrofuran, or benzene, toluene or similar aromatic solvents and the temperatures can vary from room to elevated temperatures depending on the reactivity of the reactants undergoing the reaction.

In one preferred embodiment, the process to form the ester derivative comprises “protecting” the hydroxyl groups of the ascorbic acid or derivatives thereof as esters (for example, as acetate esters) or ethers (for example, methyl ethers) or cyclic ketals and then condensing the protected ascorbic acid with the steroid halophosphate, halocarbonate or halo-oxalate under suitable reaction conditions. In general, such condensation reactions are conducted in an organic solvent such as diethyl ether, tetrahydrofuran, or benzene, toluene or similar aromatic solvents. Depending on the nature and reactivity of the reactants, the reaction temperatures may vary from low (−15° C.) to elevated temperatures.

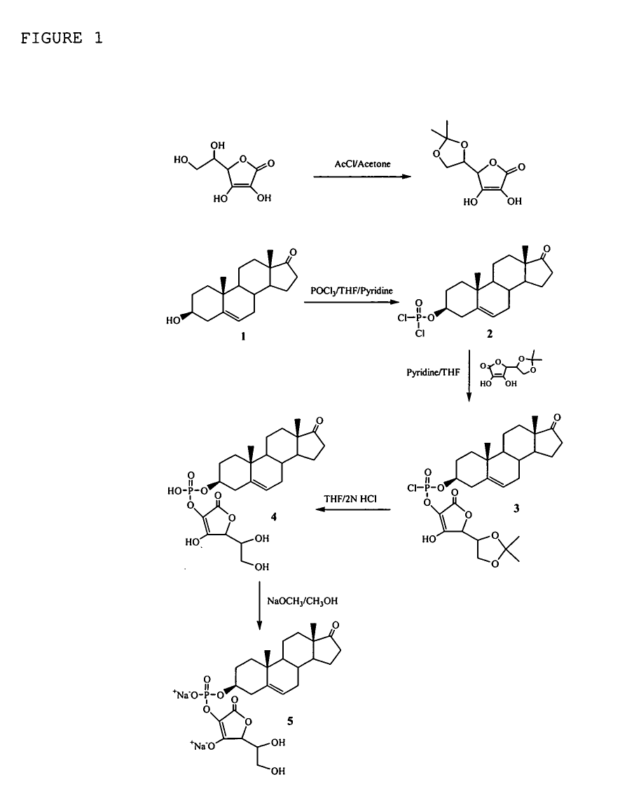

By way of example, FIG. 1 is a schematic showing the formation of the “protected” ascorbic acid (step a), the formation of the intermediary chlorophosphate/steroid derivative (step b), and the condensation reaction (steps c or d) yielding one of novel derivatives of the present invention.

In more detail, the process shown in FIG. 1 is as follows: ascorbic acid is initially converted to the cyclic ketal by the formation of 5,6-isopropylidene-ascorbic acid (shown above structure 2 in FIG. 1). This can be achieved by mixing acetone with ascorbic acid and an acid chloride under suitable reaction conditions (refer to Example 1 below). Dehydrosoandrosterone chlorophosphate is prepared by forming a solution of the steroid in anhydrous THS and pyridine (although other nitrogen bases such as aliphatic and aromatic amines may alternatively be used) and treating this solution with a phosphorus derivative such as phosphorus oxychloride. The latter suspension is then mixed with 5,6-isopropylidene-ascorbic acid in the presence of pyridine/THF at 0° C. to room temperature. Removal of the protecting group with HCL is accomplished at room temperature. After extraction, final washing and drying, the resultant novel product is ascorbyl phosphate ester of the selected steroid.

In another preferred form of the process of the present invention, ascorbic acid is protected at the hydroxyl sites not as 5,6-isopropylidene-ascorbic acid but as esters (for example as acetates, phosphates and the like.). The latter may then be condensed with the selected steroid, derivatized as described above, using known esterification methods ultimately to produce the structures of the present invention. The formation of mono and diphosphates of ascorbic acid is described thoroughly in the literature. For example, U.S. Pat. No. 4,939,128 to Kato et al., the contents of which are incorporated herein by reference, teaches the formation of phosphoric acid esters of ascorbic acid. Similarly, U.S. Pat. No. 4,999,437 to Dobler et al., the contents of which are also fully incorporated herein by reference, describes the preparation of ascorbic acid 2-phosphate. In Dobler et al., the core reaction of phosphorylating ascorbic acid or ascorbic acid derivatives with POCI3 in the presence of tertiary amines (described in German Laid Open Application DOS 2,719,303) is improved by adding to the reaction solution a magnesium compound, preferably an aqueous solution of a magnesium compound. Any of these known ascorbic acid derivatives can be used within the scope of the present invention.

The composition of the present invention encompasses not only parent “compounds” comprising the selected steroid and ascorbic acid but also the salts thereof. These salts are even more water soluble than the corresponding parent compounds and therefore their efficacy and evaluation both in vitro and in vivo will be much improved.

Salt formation of the derivatives of the present invention can be readily performed by treatment of the parent compound with a series of bases (for example, sodium methoxide or other metal alkoxides) to produce the corresponding alkali metal salts. Other metal salts of calcium, magnesium, manganese, copper, zinc, and the like can be generated by reacting the parent with suitable metal alkoxides.

Accordingly, the composition of the present invention comprises all derivatives wherein compounds within the androstane and androstene family which are coupled or linked with ascorbic acid, including all biologically acceptable salts thereof are admixed with policosanol(s) and/or policosanoic acid(s). The “linkage” between the steroid and ascorbyl moiety, thereby forming the ester, may take one or more forms described herein. Thus, the composition of the present invention may comprise all phosphate, carbonate and oxalate/steroid/ascorbyl derivatives as shown in FIGS. 1 through 6 as structures 4 and 8 and including all intermediates in the formation of these derivatives and also may comprise any halophosphate, halocarbonate and halooxalate/steroid/ascorbyl derivatives.

Examples of compounds within the scope of the present invention:

| | Chemical name, molecular |
|---|---|
| Structures | formula & formula weight |
| 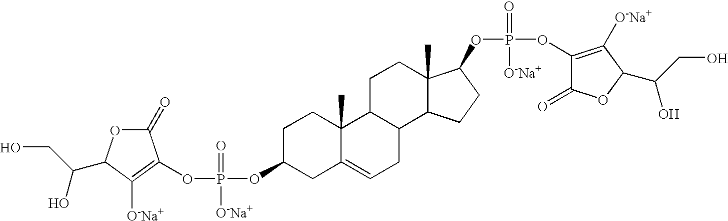 | Diascorbyl diphosphate of androst-5ene-3β,17β-diol, tetrasodium salt C₃₁H₄₀Na₄O₁₈P₂F.W 854.55 |
| 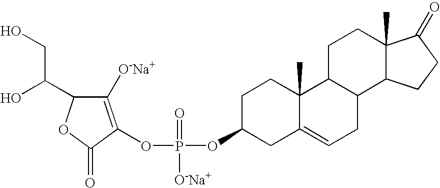 | Ascorbyl phosphate of dehydroisoandrosterone, disodium salt C₂₅H₃₃Na₂O₁₀P F.W 570.48 |
| 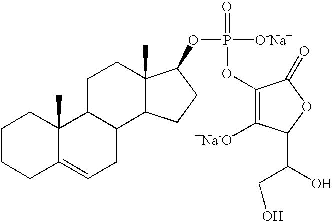 | Ascorbyl phosphate of androst-5ene-17β-ol, disodium salt C₂₅H₃₅Na₂O₉P F.W 556.49 |
| 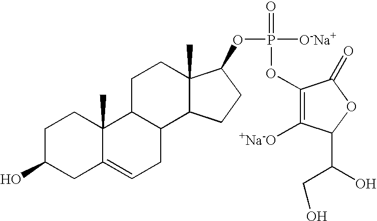 | Ascorbyl phosphate of androst-5-ene-3β,17β-diol, Disodium salt C₂₅H₃₅Na₂O₁₀P F.W 572.49 |
| 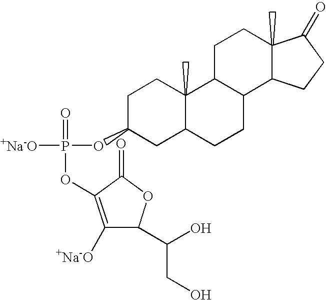 | Ascorbyl phosphate of 5α-an- droststan-3β-ol-17-one, Disodium salt C₂₅H₃₅Na₂O₁₀P F.W 572.49 |

Compositions Comprising Policosanols and/or Their Respective Acids (Policosanoic Acids) and Compound of Formulae IV:

In one aspect, the compositions of the present invention comprise one or more long chain alcohols (policosanols) and/or their respective acids (policosanoic acids) and compounds represented by formula IV, noted above. Compounds of formula IV can be prepared by known methods, for example those described below and in detail in PCT/CA00/00730, which was filed on Jun. 20, 2000 and claims priority back to U.S. patent application Ser. No. 09/339,903 filed on Jun. 23, 1999, the entire contents of which are incorporated herein by reference.

In general, compounds of formula IV, wherein the ascorbyl-based moiety is as defined in formulae V to XVI can be prepared as follows: the selected sterol or stanol (or halophosphate, halocarbonate or halo-oxalate derivatives thereof) and ascorbic acid are mixed together under reaction conditions to permit condensation of the “acid” moiety with the “alcohol” (sterol). These conditions are the same as those used in other common esterification reactions such as the Fisher esterification process in which the acid component and the alcohol component are allowed to react directly or in the presence of a suitable acid catalyst such as mineral acid, sulfuric acid, phosphoric acid, p-toluenesulfonic acid. The organic solvents generally employed in such esterification reactions are ethers such as diethyl ether, tetrahydrofuran, or benzene, toluene or similar aromatic solvents and the temperatures can vary from room to elevated temperatures depending on the reactivity of the reactants undergoing the reaction.

In a preferred embodiment, the process to form the ester comprises “protecting” the hydroxyl groups of the ascorbic acid or derivatives thereof as esters (for example, as acetate esters) or ethers (for example, methyl ethers) and then condensing the protected ascorbic acid with the sterol/stanol halophosphate, halocarbonate or halo-oxalate under suitable reaction conditions. In general, such condensation reactions are conducted in an organic solvent such as diethyl ether, tetrahydrofuran, or benzene, toluene or similar aromatic solvents. Depending on the nature and reactivity of the reactants, the reaction temperatures may vary from low (−15° C.) to elevated temperatures.

In more detail, the following is one preferred mode of preparing the compounds of formula IV, with particular reference to the ascorbyl-based moiety being as defined in one of formulae V, VI, XI or XII: ascorbic acid is initially protected from decomposition by the formation of 5,6-isopropylidene-ascorbic acid. This can be achieved by mixing acetone with ascorbic acid and an acidic catalyst such as sulfuric acid or hydrochloric acid under suitable reaction conditions. Phytostanol chlorophosphate is prepared by forming a solution of phytostanol in toluene and pyridine (although other nitrogen bases such as aliphatic and aromatic amines may alternatively be used) and treating this solution with a phosphorus derivative such as phosphorus oxychloride. The residue so formed after filtration and concentration of the mother liquor is phytostanol chlorophosphate. The latter is then mixed with 5,6-isopropylidene-ascorbic acid and, after the addition of a suitable alcohol such as ethanol and HCl, concentrated. Alternatively, pyridine/THF may be added and the product concentrated. After final washing and drying, the resultant product a stanol-phosphate-ascorbate.

In another preferred form of the process to prepare compounds of formula IV, ascorbic acid is protected at the hydroxyl sites not as 5,6-isopropylidene-ascorbic acid but as esters (for example as acetates, phosphates and the like . . . ). The latter may then be condensed with sterols or stanols, derivatized as described above, using known esterification methods ultimately to produce the compounds. The formation of mono and diphosphates of ascorbic acid is described thoroughly in the literature. For example, U.S. Pat. No. 4,939,128 to Kato et al., the contents of which are incorporated herein by reference, teaches the formation of phosphoric acid esters of ascorbic acid. Similarly, U.S. Pat. No. 4,999,437 to Dobler et al., the contents of which are also fully incorporated herein by reference, describes the preparation of ascorbic acid 2-phosphate. In Dobler et al., the core reaction of phosphorylating ascorbic acid or ascorbic acid derivatives with POCI3 in the presence of tertiary amines (described in German Laid Open Application DOS 2,719,303) is improved by adding to the reaction solution a magnesium compound, preferably an aqueous solution of a magnesium compound. Any of these known ascorbic acid derivatives can be used.

In more detail, the following is another preferred mode of preparing the compounds of formula IV and in particular wherein the ascorbyl-based moiety is represented by formulae VII, IX, XIII and XV: prepare the “protected” ascorbic acid and follow the same process outlined in detail above; however, the phosphorus oxylchloride is replaced by oxalyl chloride thereby yielding a stanol-oxalate-ascorbate.

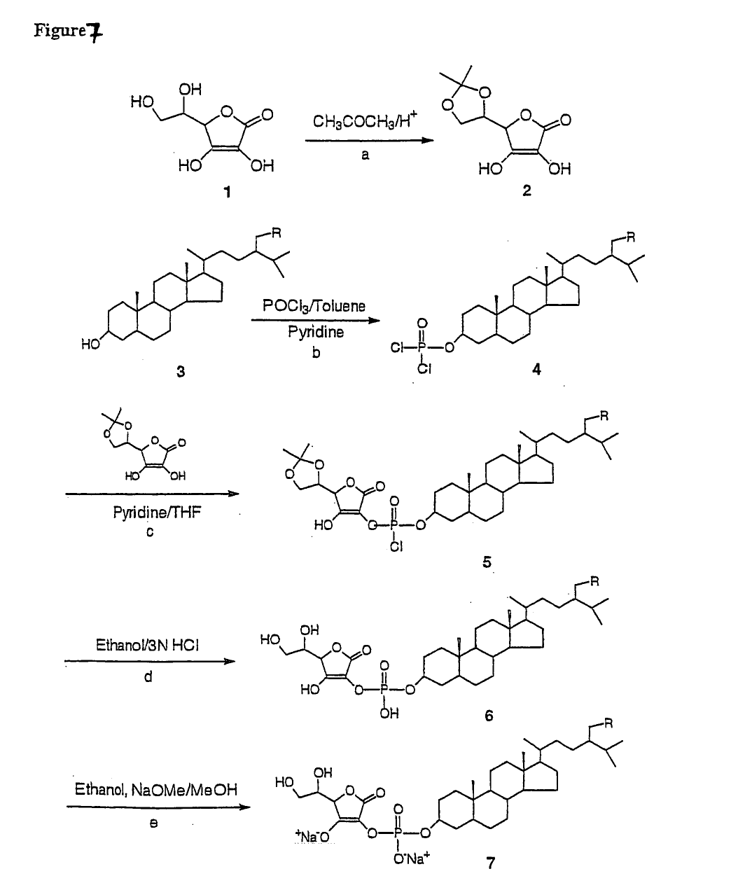

FIG. 7 is a schematic showing the formation of the “protected” ascorbic acid (step a), the formation of the intermediary chlorophosphate/stanol derivative (step b), and the condensation reaction (alternatively steps c or d) yielding one of novel derivatives of the: phytostanol-phosphate-ascorbate (noted as structure 6).

In more detail, the process shown in FIG. 7 is as follows: ascorbic acid is initially protected from decomposition by the formation of 5,6-isopropylidene-ascorbic acid (structure 2). This can be achieved by mixing acetone with ascorbic acid and an acidic catalyst such as sulfuric acid or hydrochloric acid under suitable reaction conditions (refer to examples). Phytostanol chlorophosphate (structure 4) is prepared by forming a solution of phytostanol in toluene and pyridine (although other nitrogen bases such as aliphatic and aromatic amines may alternatively be used) and treating this solution with a phosphorus derivative such as phosphorus oxychloride. The residue so formed after filtration and concentration of the mother liquor is phytostanol chlorophosphate (structure 4). The latter is then mixed with 5,6-isopropylidene-ascorbic acid and, after the addition of a suitable alcohol such as ethanol and HCl (step d), concentrated. Alternatively, pyridinefTHF may be added (step c) and the product concentrated. After final washing and drying (step e), the resultant novel product of both steps c or d is phytostanol-phosphate-ascorbate (structure 6).

As noted above, in another preferred form of the process of the present invention, ascorbic acid is protected at the hydroxyl sites not as 5,6-isopropylidene-ascorbic acid but as esters (for example as acetates, phosphates and the like.). The latter may then be condensed with phytosterols or phytostanols, derivatized as described above, using known esterification methods ultimately to produce the structures of the present invention. The formation of mono and diphosphates of ascorbic acid is described thoroughly in the literature.

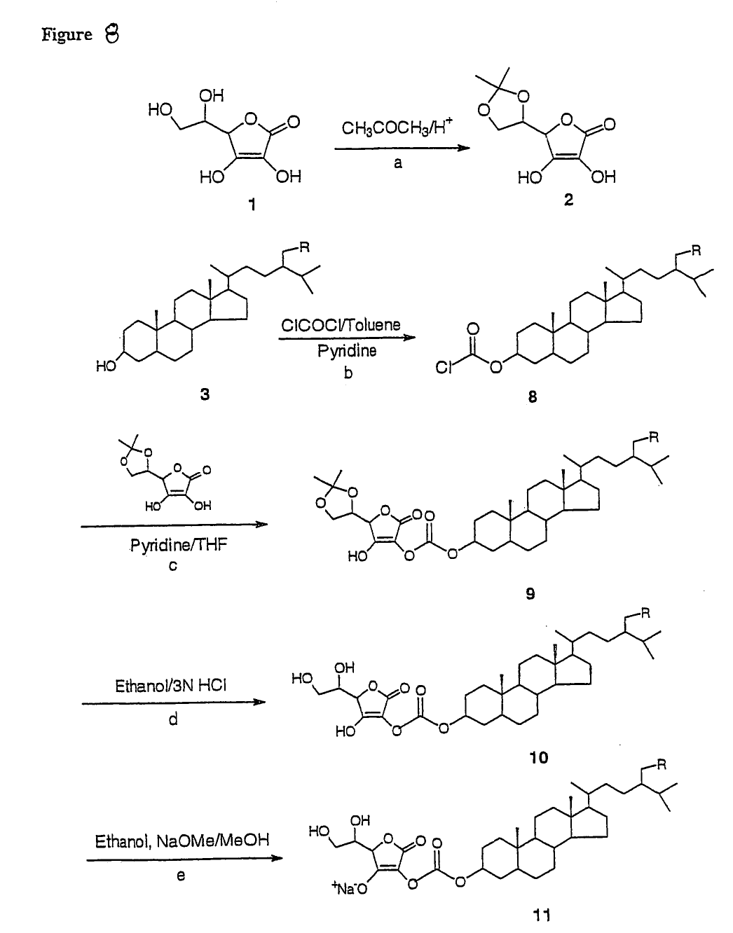

FIG. 8 is a schematic showing the formation of the “protected” ascorbic acid (step a), the formation of the intermediary chlorocarbonate/stanol derivative (step b), and the condensation reaction (optionally steps c or d) yielding structure 9 (10 is the same), one of novel derivatives of the present invention: phytostanol-carbonate-ascorbate. These chlorocarbonate derivatives may be prepared by the same process outlined in detail above with respect to FIG. 7; however, the phosphorus oxylchloride is replaced (as shown in step b of FIG. 8) by phosgene.

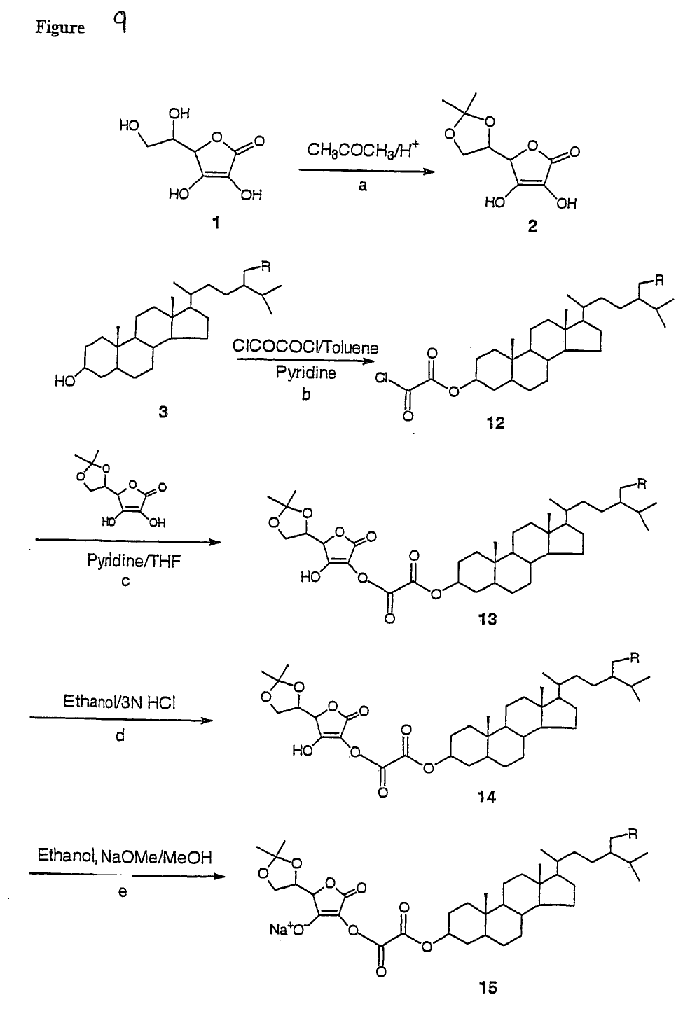

FIG. 9 is a schematic showing the formation of the “protected” ascorbic acid (step a), the formation of the intermediary chloro-oxalate/stanol derivative (step b), and the condensation reaction (optionally steps c or d) yielding a novel structure 13 (same as 14), one of novel derivatives of the present invention: phytostanol-oxalate-ascorbate (noted as structure 14). These chloro-oxalate derivatives may be prepared by the same process outlined in detail above with respect to FIG. 7; however, the phosphorus oxylchloride is replaced (as shown in step b of FIG. 9) by oxalyl chloride.

Preferably, in the compounds of formula IV, the sterol or stanol moiety is naturally-derived or artificially synthesized beta-sitosterol, campestanol, sitostanol, and campesterol. In the most preferred form, formula IV comprises a chemical linkage between one or more disodium ascorbyl phytostanyl phosphates (referred to herein as “FM-VP4”) which comprises two major components: disodium ascorbyl campestanyl phosphate (“DACP”) and disodium ascorbyl sitostanyl phosphate (“DASP”). In order words, a preferred form of composition of the present invention comprises a mixture of DACP, DASP and one or more policosanols and/or policosanoic acids.

Salts

As used herein, the term “biologically acceptable salts” refers any salts that retain the desired biological and/or physiological activity of the compounds as described herein and exhibit minimal undesired toxicological effects. Accordingly, reference to all compounds herein thereby includes reference to respective acidic and/or base salts thereof, formed with inorganic and/or organic acids and bases.

Exemplary acid addition salts include acetates (such as those formed with acetic acid or trihaloacetic acid, for example trifluroacetic acid), adipates, alginates, ascorbates, aspartates, benzoates, benzenesulfonates, bisulfates, borates, butyrates, citrates, camphorates, camphorsulfonates, cyclpentanepropionates, digluconates, dodecylsulfates, heptanoates, hexanoates, hydrochlorideshyrobromides, hydroiodides, 2-hydroethanesulfonates, lactates, maleates, methanesulfonates, 2-naphthalenesulfonates, nicotinates, nitrates, oxalates, pectinates, persulfonates, 3-phenylpropionates, phosphates, picrates, pivalates, propionates, salicylates, succinates, sulfates, sulfonates, tartrates, thiocyantes, toluenesulfonates, undecanoates and the like.

Those compounds which contain an acid moiety may form salts with a variety or organic and inorganic bases.

With respect to the formation of these derivatives, it is to be appreciated that, while selected synthesis processes are described, there are a number of other means by which the variety of derivatives disclosed and claimed can be made. It is well within the purview of a skilled person in this chemical field, once a particular derivative is chosen, to undertake the synthesis using commonly available techniques in the art. For this reason, the complete synthesis of each and every claimed derivative is not described.

To the extent that the compounds as described herein and salts thereof may exist in their tautomeric form, all such tautomeric forms are contemplated herein as part of the present invention.

All stereoisomers of the compounds described herein, such as those which may exist due to asymmetric carbons on various constituents, including enantiomeric forms (which may exist even in the absence of asymmetric carbons) and diastereomeric forms, are contemplated within the scope of the present invention. Individual stereoisomers of the compounds described herein may, for example, be admixed as racemates or with all other, or other selected stereoisomers. The chiral centres of the compounds can have the S or R configuration as defined by the IUPAC 1974 Recommendations. Such stereoisomers can be prepared using conventional techniques, either by reacting enantiomeric starting materials, or by separating isomers of compounds of the present invention. When diastereomeric or enantomeric products are prepared, they can be separated by conventional methods, for example, chromatographic or fractional crystallization.

Isomers may include geometric isomers, for example cis-isomers or trans-isomers across a double bond. All such isomers are contemplated among the compounds useful herein. The compounds useful in the present invention also include tautomers.

Uses and Advantages of Compositions

What is achieved within the scope of the present invention is the creation of a new composition, comprising two moieties with dual complementary effects, particularly with respect to cholesterol lowering. It is suspected that at least part of this effect is due to the distinct yet complementary mechanism of actions of, on the one hand, the sterol and steroid moiety, which forms part of formulae I through IV and, on the other hand, the policosanol moiety.

The union benefits and enhances the both parts of this new composition. The sterol or steroid moiety, formerly poorly soluble, becomes, as part of an ascorbyl-based compound, much more readily soluble in aqueous and non-aqueous media such as oils and fats. Accordingly, administration of the sterol or steroid moiety becomes possible without any further enhancements to modify its delivery. Furthermore, with respect to lowering LDL cholesterol, there is an unexpected complementary effect between the respective components.

In accordance with the present invention, it has been surprisingly discovered that the compositions described herein have enormous potential in various pharmacological fields while obviating many of the limitations of using the components alone. In particular, the present invention provides methods for treating and/or preventing a plurality of diseases, conditions and disorders.

According to one aspect of the present invention, there is provided a method for treating and/or preventing CVD and its underlying manifestations including atherosclerosis, hypercholesterolemia, hyperlipidemia, hypertension, thrombosis, coronary artery disease, aneurysm, myocardial infarction, embolism, stroke, thrombosis, angina or unstable angina, coronary plaque inflammation, related diseases such as Type II diabetes, as well as treating diseases, conditions or disorders in which immune function is compromised or in which immune system enhancement is required, including radiation-related injuries, HIV, AIDS, hepatitis, chronic fatigue syndrome, and malaria, as well as reducing inflammation, caused by, for example bacterial-induced inflammation, viral-induced inflammation, chronic inflammatory bowel disease and inflammation associated with surgical procedures and injury, as well as being useful to control weight gain or promote weight loss, as well as being useful in preventing cancer, as well as exhibiting anti-aging effects by administering to an animal, particularly a human, a composition comprising one or more long chain alcohols (policosanols) and/or their respective acids (policosanoic acids) and one or more ascorbic acid derivatives, having one or more of the formulae I, II and III or III.

According to another aspect of the present invention, there is provided a method for treating and/or preventing CVD and its underlying manifestations including atherosclerosis, hypercholesterolemia, hyperlipidemia, hypertension, thrombosis, coronary artery disease, coronary plaque inflammation, as well as exhibiting anti-oxidant effects by administering to an animal, particularly a human, a composition comprising one or more long chain alcohols (policosanols) and/or their respective acids (policosanoic acids) and one or more ascorbic acid derivatives, having the formula IV.

The desired effects described herein may be achieved in a number of different ways. The compounds of formulae I to IV (A) and the policosanol/policosanoic acid moiety (B) may be administered by any conventional means available for use in conjunction with pharmaceuticals for example, with pharmaceutically acceptable carriers, additives, adjuvants or excipients. Generally, however, the pharmaceutical compositions comprising A and B comprise from about 1% to 99% by weight of these “active” components and preferably from about 5% to 95% by weight of the active components.

It is preferred that the composition of the present invention comprise from 10% to 80% by weight A and from 1% to 70% by weight B, more preferably from 20% to 70% by weight A and from 10% to 40% by weight B. In a further preferred embodiment, the ratio of A to B, by weight, is from 1:1 to 50:1, preferably 2:1 to 20:1.

The formulations and pharmaceutical compositions can be prepared using conventional, pharmaceutically available excipients, and additives and by conventional techniques. Such pharmaceutically acceptable excipients and additives include non-toxic compatible fillers, binders, disintegrants, buffers, preservatives, anti-oxidants, lubricants, flavourings, thickeners, colouring agents, emulsifiers and the like.

The exact amount or dose of A and B which is required to achieve the desired effects will, of course, depend on a number of factors such as the particular compound or composition chosen, the disease, condition or disorder sought to be treated or prevented, the result to be achieved, the potency of the compound or composition administered, the formulation in which it is administered, the mode of administration, and the age, weight, condition and response of the patient as well as other factors known to those skilled in the art of food additives and medicinal agents. All of these factors, among others, will be considered by the attending clinician with respect to each individual or patient.

Generally, within the composition described herein, a total daily dose of A having one of formulae I to IV may be administered in a daily dosage range of from 10 mg to about 20 g, more preferably 10 mg to 1.5 g, and most preferably up to 800 mg per day in single or multiple divided doses.

It should be recognized that the provision of much larger daily doses of the compounds of formulae I to IV and their derivatives are not harmful to the animal host, as excess will simply pass through normal excretory channels.

With respect to B, the policosanols and/or policosanoic moiety, and within the composition described herein, it is preferred to provide from 1-200 mg/day, preferably from 5 to 100 mg/day, more preferably from 5 to 20 mg/day. At even significantly higher doses than these, policosanols and policosanoic acids have proven to be safe. It is to be understood that generally, the higher the respective octacosanol and/or octacosanoic acid component in the composition, the lower the amount required for therapeutic efficacy.

Delivery Systems

Although it is fully contemplated within the scope of the present invention that the compositions may be administered to animals, particularly humans, directly and without any further modification, it is possible to take further steps to enhance delivery and ensure even distribution throughout the food, beverage, pharmaceutical, nutraceutical and the like to which they are added. It is to be understood; however, that these steps are purely optional. Such enhancement may be achieved by a number of suitable means such as, for example, solubilizing or dispersing the components of the composition to form emulsions, solutions and dispersions or self-emulsifying systems; lyophilizing, spray drying, controlled precipitating, or a combination thereof; forming solid dispersions, suspensions, hydrated lipid systems; forming inclusion complexations with cyclodextrins; and using hydrotopes and formulations with bile acids and their derivatives. Alternatively, and optionally in conjunction with any one of these solubility and/or dispersability enhancement methods, the compositions may be incorporated into various vehicles in order to achieve the therapeutic objectives set out herein.

Without limiting the generality of the foregoing, the compositions of the present invention may be admixed with various carriers, excipients or adjuvants to assist in direct administration or to assist in the incorporation of the composition into foods, beverages, nutraceuticals or pharmaceuticals. In order to appreciate the various possible vehicles of the delivery of the derivatives, the list below is provided.

1) Pharmaceutical Dosage Forms:

It is contemplated within the scope of the present invention that the compositions of the present invention may be incorporated into various conventional pharmaceutical preparations and dosage forms such as, for example, tablets (plain and coated), including chewable and fast melt tablets, for use orally, bucally or lingually, capsules (hard and soft, gelatin, with or without additional coatings), capsules including two piece hard shell capsules (gelatin, modified cellulose or other appropriate biopolymers) and soft shell capsules (gelatin, modified cellulose or other appropriate biopolymers), powders, granules (including effervescent granules), pellets, microparticulates, suspensions, solutions (such as micellar, syrups, elixirs and drops), lozenges, pastilles, ampoules, emulsions, microemulsions, ointments, creams, suppositories, gels, transdermal patches and modified release dosage forms together with customary excipients and/or diluents and stabilizers.

The compositions of the present invention, adapted into the appropriate dosage form, may be administered to animals, including humans, orally, by injection (intravenously, subcutaneously, intra-peritoneally, intra-dermally or intramuscularly), topically or in other ways.

The compositions of the present invention can be administered to a patient either by themselves, or in pharmaceutical compositions where they are mixed with suitable carriers or excipients.

Use of pharmaceutically acceptable carriers to formulate the compositions herein disclosed for the practice of the invention into dosages suitable for systemic administration is within the scope of the invention. With proper choice of carrier and suitable manufacturing practice, the compositions of the present invention, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the invention to be formulated as tablets, pills, capsules, liquids, gels, syrups, slurries, suspensions and the like, for oral ingestion by a patient to be treated.

Pharmaceutical vehicles, comprising one or more of the compositions of the present invention, include compositions wherein the active ingredients are contained in an effective amount to achieve their intended purpose. Determination of the effective amounts is well within the capability of those skilled in the art, especially in light of the detailed disclosure provided herein.

In addition to the active ingredients these pharmaceutical compositions may contain suitable pharmaceutically acceptable carriers comprising excipients and auxiliaries which facilitate processing of the active compounds into preparations which can be used pharmaceutically. The preparations formulated for oral administration may be in any suitable, biologically acceptable form, including those forms described herein.

The pharmaceutical compositions of the present invention may be manufactured in a manner that is itself known, e.g., by means of conventional mixing, dissolving, granulating, dragee-making, levigating, emulsifying, encapsulating, entrapping or lyophilizing processes.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

In one preferred form, the composition of the present invention is administered in the form of a liposome. Liposomes are hollow microspheres composed of one or more double lipid layers. They were first used more than 30 years ago as vehicles for various drug substances, and since then knowledge of their behavior in vitro has allowed a more rational design focused on the specific treatment of certain diseases.

Formation of liposomes occurs formed when thin lipid films are hydrated. The hydrated lipid sheets detach during agitation and self-close to form multi-lamellar vesicles. There are a number of methods known and widely practiced in the in the art to prepare liposomes. Chemotherapeutic agents, such as doxorubicin, are often encapsulated in liposomes using the established methods.

Typically, 100 nm diameter liposomes are prepared by exposing chloroformic solution of various lipid mixtures to high vacuum and subsequently hydrating the resulting lipid films (DSPC/CHOL, EPC/CHOL, DSPC/PEG-PE/CHOL) with pH 4 buffers, and extruding them through polycarbonated filters, after a freezing and thawing procedure. A transmembrane pH gradient is then created by adjusting the pH of the extravesicular medium to 7.5 by addition of an alkalinization agent. The technique exploits the ability of weak bases to redistribute across membranes exhibiting pH gradients where [drug]in/[drug]out=[H+]in/[H+]out. The selected drug is then entrapped by addition of the drug solution in small aliquots to the vesicle solution, at an elevated temperature, to allow drug accumulation inside the liposomes. Trapping efficiencies are determined by separating free from liposome encapsulated drug on gel filtration columns and quantifying the two fractions for lipid and drug content by liquid scintillation counting, fluorescence spectroscopy or UV-VIS spectroscopy. These liposomes are then evaluated for size distribution (quasielastic light scattering, scanning electron microscopy), drug uptake and release studies, stability, and in vivo tumor targeting efficiency.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

Oral liquid preparations may be in the form of, for example, emulsions, syrups, or elixirs, or may be presented as a dry product for reconstitution with water or other suitable vehicle before use. Such liquid preparations may contain conventional additives such as suspending agents, for example sorbitol, syrup, methyl cellulose, gelatin, hydroxyethylcellulose, carboxymethylcellulose, aluminium stearate gel, hydrogenated edible fats; emulsifying agents, for example lecithin, sorbitan monooleate, or acacia; non-aqueous vehicles (which may include edible oils), for example almond oil, fractionated coconut oil, oily esters such as esters of glycerine, propylene glycol, or ethyl alcohol; preservatives, for example methyl or propyl p-hydroxybenzoate or sorbic acid; and if desired conventional flavouring or colouring agents.

Without further elaboration, the foregoing so fully illustrates the present invention that others may, by applying current or future knowledge, adapt the same for use under the various conditions described and claimed herein.

### EXAMPLES

The present invention is illustrated, but not limited, by the following examples:

### Example 1

### Preparation of Compounds of Formulae I-III

### Protection of Ascorbic Acid and Synthesis of Disodium Ascorbyl Phosphate Ester of Dehydroisoandrosterone

To a dry round bottom flask, acetone (150 ml) and L-ascorbic acid (50 g) were added at 0° C. Acetyl chloride (7.5 ml) was added dropwise through an addition funnel in 10 minutes. The reaction mixture was stirred at 0° C. for 24 hours. The precipitate was filtered off and washed with acetone (3×20 ml). The white product, 5,6-isopropylidine ascorbic acid, was dried under vacuum for 1.5 hours to give a dry powder (52 g), yield 85%.

A dry three neck round bottom flask was fitted with a stirring bar, argon inlet and an addition funnel. A solution of dehydroisoandrosterone (FIG. 1, 1.73 g, 6 mmol) in anhydrous THF (15 ml) and pyridine (2.4 ml) was added dropwise to the mixture of anhydrous THF (12 ml) and POCl₃ (0.7 ml, 7.5 mmol) at 0° C. over a period of 10 minutes. A white precipitate formed immediately. The suspension was stirred at 0° C. for 40 minutes, and at room temperature for 1 hour and 40 minutes.

To the above suspension, a solution of 5,6-isopropylidine ascorbic acid (3.6 g, 16.67 mmol) in anhydrous pyridine (3 ml) and THF (30 ml) was added dropwise at 0° C. over a period of 20 minutes. The suspension was stirred at 0° C. for 30 minutes, and at room temperature for 1.5 hours. The formed pyridinium chloride was filtered out and washed with THF twice. The solvents were evaporated under reduced pressure at 40° C. to afford a residue (3, FIG. 1).

The residue (3, Scheme 1) was dissolved in THF (40 ml), and 2N HCl (30 ml) was added in one portion. The mixture was stirred at room temperature for 8 hours. THF was evaporated under a reduced pressure. The water layer was extracted with ethyl acetate (4×50 ml). The combined ethyl acetate solution was washed with brine (100 ml), and dried over Na₂SO₄. The solvent was evaporated to give a residue. The residue was dissolved in CHCl₃, and then hexanes was added to precipitate the product. The precipitated solid was filtered out, washed with hexanes and dried under vacuum (2.43 g, crude product, yield: 77%). The purification of phosphate ester was done by reverse phase C-18 chromatography (Waters, water/methanol=90/10 to 60/40). Pure compound 4 (FIG. 1, 39 mg) was isolated from 50 mg of the crude product. The overall yield (base on dehydroisoandrosterone) was 60%.

Ascorbyl phosphate ester of dehydroisoandrosterone (4, Scheme 1, 0.5 g, 0.95 mmol) was dissolved in methanol (3 ml) at room temperature, and then sodium methoxide in methanol (1 ml, 20%) was added. The suspension was stirred at room temperature for 30 minutes. The precipitated solid was filtered out, washed with methanol, acetone and hexanes. The mother liquor was concentrated to 2 ml, acetone was added to precipitate the product. An additional white solid was obtained. The combined solid was dried under vacuum at room temperature. Disodium ascorbyl phosphate ester of dehydroisoandrosterone (5, FIG. 1, 0.49 g, yield 91%) was obtained.

### Example 2

### Preparation of Compounds of Formulae I-III

### Synthesis of Disodium Ascorbyl Phosphate Ester of 5α-Androstan-3β-ol-17-one

To a dry round bottom flask, 5α-androstan-3β-ol-17-one (1.0 g, 3.4 mmol), THF (8.6 ml) and pyridine (1.38 ml) were added. The mixture was stirred at room temperature until a clear solution was obtained. To another dry round bottom flask, THF (6.9 ml) and POCl₃ (0.4 ml, 4.25 mmol) were added, stirred at 0° C. for 5 minutes. To this mixture, the above prepared 5α-androstan-3β-ol-17-one solution was added drop-wise under argon atmosphere over a period of 10 minutes. After the addition, the white suspension was stirred at 0° C. for 35 minutes, and at room temperature for 2 hours. The reaction was stopped and the white suspension was used for the coupling reaction without filtration.

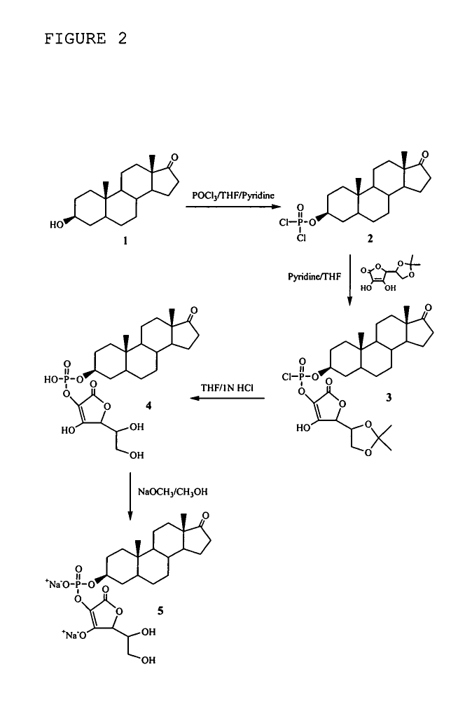

5,6-Isopropylidine ascorbic acid (2.0 g, 9.52 mmol) was dissolved in pyridine (1.71 ml) and THF (17 ml). The round bottom flask which contained previously prepared white suspension (2, FIG. 2) was immersed in an ice-water bath. To this mixture, the above prepared THF solution of the 5,6-isopropylidine ascorbic acid was added dropwise under stirring at 0° C. over a period of 15 minutes. After the addition, the mixture was stirred at 0° C. for 25 minutes, and at room temperature for 2 hours. The white solid of pyridinium chloride was filtered out and washed with THF (8 ml). The filtrate was concentrated to remove THF and excess pyridine to give a residue (3, FIG. 2, 2.38 g).

The residue (3, Figure) was dissolved in THF (30 ml), and 1N HCl (30 ml) was added in one portion. The mixture was stirred at room temperature for 16 hours and 45 minutes. 12N HCl (4 ml) was added to the reaction mixture at room temperature. The reaction mixture was stirred at room temperature for an additional 4 hours and 45 minutes. THF was evaporated under a reduced pressure. The water layer was extracted with ethyl acetate (3×60 ml). The combined ethyl acetate solution was washed with brine (60 ml), and dried over Na₂SO₄. The extract was concentrated to about 3 ml. Hexanes (15 ml) was added to precipitate the product. The precipitated solid was filtered out, washed with hexanes and dried under a reduced pressure (1.48 g, 4, FIG. 2).

Ascorbyl phosphate ester of 5α-androstan-3β-ol-17-one (4, FIG. 2, 0.5 g, 0.95 mmol) was dissolved in methanol (3 ml) at room temperature, and then sodium methoxide in methanol (1.5 ml, 20%) was added. The suspension was stirred at room temperature for 25 minutes. The precipitated solid was filtered out, washed with methanol, acetone and hexanes. The mother liquid was concentrated to 2 ml, and then acetone was added to precipitate the product. An additional product was obtained. The combined solid was dried under a reduced pressure at room temperature to give disodium ascorbyl phosphate ester of 5α-androstan-3β-ol-17-one (5, FIG. 2, 0.38 g). The overall yield was 57% (based on 5α-androstan-3β-ol-17-one).

### Example 3

### Preparation of Compounds of Formulae I-II Synthesis of Disodium Ascorbyl Phosphate Ester of Androst-5-ene-3β,17β-diol

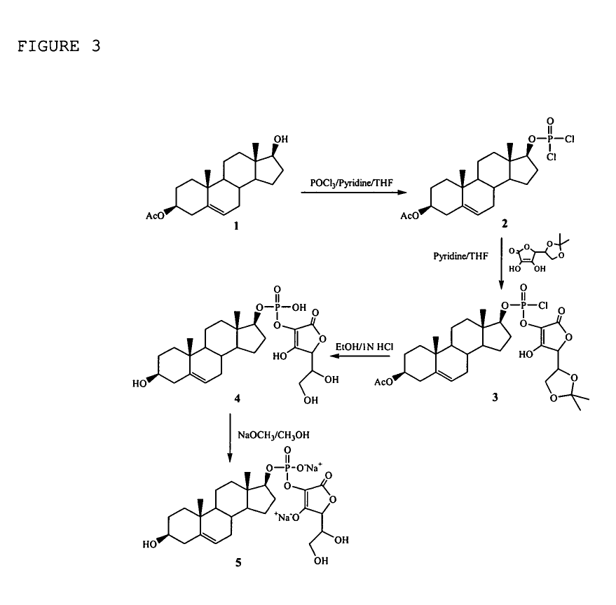

To a dry round bottom flask, 3β-acetoxyandrost-5-ene-17β-ol (1, FIG. 3, 1.0 g, 3.0 mmol), anhydrous THF (6.3 ml) and pyridine (0.73 ml) were added. The mixture was stirred at room temperature until a clear solution was obtained. To another dry round bottom flask, THF (2 ml) and POCl₃ (0.35 ml, 3.22 mmol) were added, stirred at −5° C.˜-10° C. for 5 minutes. To this mixture, the above prepared 3β-acetoxyandrost-5-ene-17β-ol solution was added drop-wise under argon atmosphere over a period of 20 minutes. After the addition, the white suspension was stirred at room temperature for 1 hour. The mixture was concentrated to remove THF and excess POCl₃ to give a residue (2, FIG. 3).

5,6-Isopropylidine ascorbic acid (0.98 g, 4.55 mmol) was dissolved in anhydrous pyridine (0.70 ml) and THF (6.2 ml). The residue (2, FIG. 3 dissolved in dry THF (4 ml). To this mixture, the above prepared THF solution of the 5,6-isopropylidine ascorbic acid added dropwise under stirring at 0° C. over a period of 20 minutes. After the addition, the mixture was stirred at room temperature for 1 hour and 25 minutes. The white solid of pyridinium chloride was filtered out and washed with THF (6 ml). The filtrate was concentrated to remove THF and excess pyridine to give a residue (3, FIG. 3).

The residue (3, FIG. 3) was dissolved in a mixture of ethanol (12.5 ml) and 1N HCl (12.5 ml). The mixture was kept stirring at 50° C.˜55° C. for additional 3 hours and 45 minutes (TLC monitoring). The mixture was extracted with ethyl acetate (60 ml), washed with 10% aqueous NaCl twice (30 ml, 20 ml) and dried over Na₂SO₄ (10 g) for 1.5 hours. After the filtration, the filtrate was concentrated to 5 ml. Hexanes (10 ml) was added to precipitate the product. The precipitate was collected, washed with hexanes (10 ml) and dried under the reduced pressure to give a slightly yellow powder (4, FIG. 3, 0.95 g, crude product, yield 60%). The pure product was obtained by preparative HPLC.

Instrument is Waters Delta Preparative 4000 HPLC system. Column is Waters Symmetry C18, 5 μm, 30×100 mm. Mobile phases are 0.1% H₃PO₄ in water and acetonitrile. Water and acetonitrile are HPLC grade or equivalent.

The crude product was purified by preparative HPLC. The product was collected and evaporated on a rotary evaporator to remove acetonitrile. The water solution was extracted with ethyl acetate twice. The ethyl acetate layer was dried over Na₂SO₄, concentrated and dried under a reduced pressure to give a white powder product. This product was submitted for NMR and mass spectra. Both spectra indicated the product is ascorbyl phosphate ester of androst-5-ene-3β,17β-diol (4, Figure).

Preparation of disodium ascorbyl phosphate ester of androst-5-ene-3β,17β-diol (5, FIG. 3) was similar to the process described in Example 2.

### Example 4

### Preparation of Compounds of Formulae I-III

### Synthesis of Disodium Ascorbyl Phosphate Ester of Androst-5-ene-17β-ol

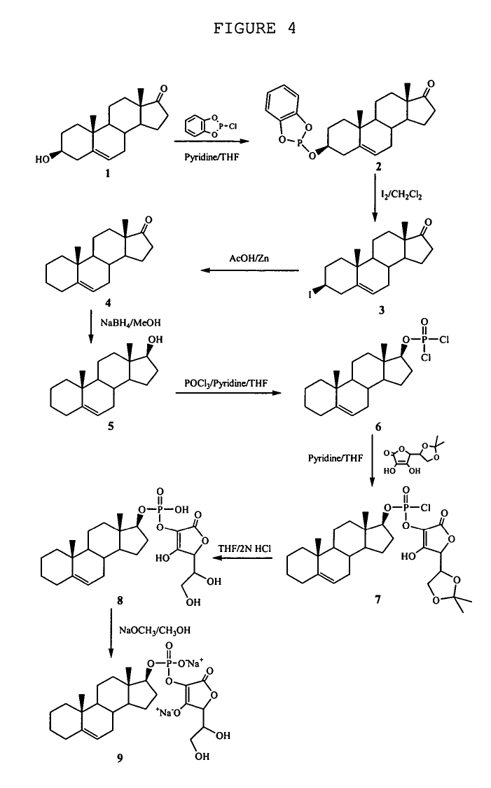

To a solution of pyridine (0.41 ml) and 1,2-phenylenephosphorochloridite (0.6 ml, 5 mmol) in anhydrous THF (10 ml) at 0° C. was added dropwise dehydroisoandrosterone (1, FIG. 4, 1.44 g, 5 mmol) in anhydrous THF (10 ml) over a period of 10 minutes. The reaction mixture was stirred at 0° C. for 30 minutes, and at room temperature for 4 hours. The reaction was monitored with TLC (hexanes/EtOAc=2/1). The formed pyridinium chloride was filtered off and washed with THF. The solvents were evaporated at 40° C. to give a white powder (2, FIG. 4).

The crude phosphite ester (2, FIG. 4) was dissolved in methylene chloride (25 ml), and treated with iodine (1.27 g) for 4 hours at room temperature. The reaction mixture was diluted with methylene chloride (75 ml), washed with 1N NaOH (2×50 ml) and water (2×50 ml), and dried over Na₂SO₄. The solvent was removed, and the product (3, Scheme 4, 1.4 g, yield 71%) was crystallized from methylene chloride and methanol.

3β-Iodoandrost-5-ene-17-one (3, FIG. 4, 1.27 g, 3.19 mmol) was dissolved in glacial acetic acid (40 ml) at 50-55° C., the activated zinc dust (2.7 g) was added in one portion. The mixture was stirred at 50° C.-55° C. for 2 hours, the zinc dust was filtered out and washed with methylene chloride. The solution was diluted with methylene chloride (120 ml), washed with water (2×100 ml), 1N NaOH (2×100 ml) and water (100 ml), and dried over Na₂SO₄. The solvent was removed to afford a white powder. The white powder was dried under vacuum to give androst-5-ene-17-one (4, FIG. 4, 0.83 g, yield: 95%).

Androst-5-ene-17-one (4, FIG. 4, 0.65 g, 2.34 mmol) was dissolved in methanol (25 ml) at room temperature. The solution was cooled down to 0° C., and NaBH₄ (50 mg) was added in one portion. The mixture was stirred at 0° C. for 3 hours, and monitored with TLC (hexanes/EtOAc=3/1). After 3 hours, another portion of NaBH₄ (20 mg) was added, and the reaction mixture was stirred at 0° C. for additional half an hour. Aqueous NH₄Cl (5%, 25 ml) and HCl (6N, 5 ml) were added slowly at 0° C., and stirred for 1 hour. Water (100 ml) was added to completely precipitate the product. The precipitated solid was filtered out and washed with water, and dried under vacuum. The pure product (5, FIG. 4, 0.62 g, yield: 95%) was obtained by column chromatography.

A solution of androst-5-ene-17β-ol (5, FIG. 4, 0.63 g, 2.3 mmol) in anhydrous THF (8 ml) and pyridine (1 ml) was added drop-wise to the mixture of anhydrous THF (6 ml) and POCl₃ (0.28 ml, 3 mmol) at 0° C. over a period of 5 minutes. The suspension was stirred at 0° C. for 50 minutes, and then at room temperature for one hour (6, FIG. 4).

To the above suspension, a solution of 5,6-isopropylidine ascorbic acid (1.38 g) in anhydrous pyridine (1.2 ml) and THF (12 ml) was added drop-wise at 0° C. over a period of 15 minutes. The suspension was stirred for 1.5 hours at 0° C., and then overnight at room temperature. The formed pyridine hydrochloride was filtered out and washed with THF twice. The solvents were evaporated under reduced pressure at 40° C. to afford a residue (7, FIG. 4).

The residue (7, FIG. 4) was dissolved in THF (35 ml), and 2N HCl (30 ml) was added as one portion. The mixture was stirred overnight at room temperature. THF was evaporated under reduced pressure. The water layer was extracted with ethyl acetate (3×100 ml). The combined ethyl acetate solution was washed with brine (100 ml), and dried over Na₂SO₄. The solvent was evaporated to give a residue. The residue was dissolved in acetone, and hexanes was added to precipitate the product. The white precipitated solid was filtered out, washed with hexanes and dried under vacuum (8, FIG. 4, 0.82 g, crude product, yield: 70%).

Preparation of disodium ascorbyl phosphate ester of androst-5-ene-1 7β-ol was similar to example 1.

### Example 5

### Preparation of Compounds of Formulae I-III

### Synthesis of Tetra-sodium Monoascorbyl Diphosphate Ester of 3β-Acetoxyandrost-5-ene-7β,17β-diol

To a dry round bottom flask, 3β-acetoxyandrost-5-ene-7β,17β-diol (0.5 g, 1.43 mmol), pyridine (0.83 ml) and THF (4 ml) were added. The mixture was stirred at room temperature until a clear solution was obtained. To another dry round bottom flask, THF (5 ml) and POCl₃ (0.33 ml) were added, stirred at −5° C.˜0° C. for 5 minutes. To this mixture, the above prepared 3β-acetoxyandrost-5-ene-7β,17β-diol solution was added dropwise under argon atmosphere over a period of 15 minutes. After the addition, the white suspension was stirred at room temperature for 2 hours and 45 minutes. The reaction was stopped and the white suspension was used for the coupling reaction without filtration.

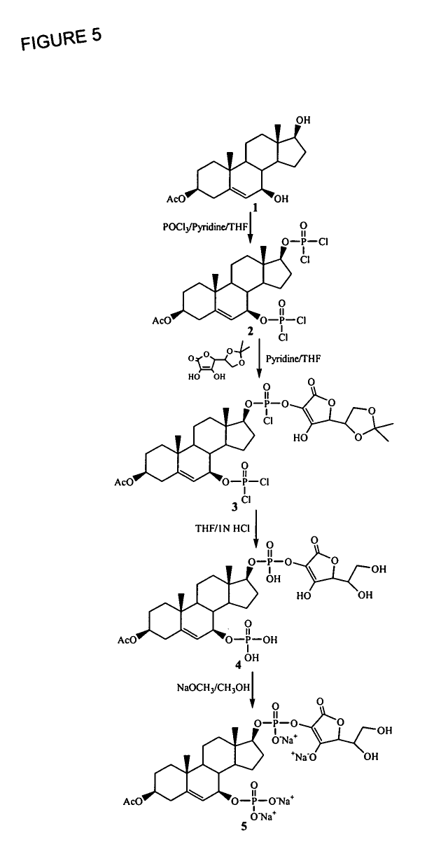

5,6-Isopropylidine ascorbic acid (1.30 g, 6.02 mmol) was dissolved in pyridine (1.16 ml) and THF (5.8 ml). The round bottom flask which contained previously prepared white suspension (2, FIG. 5) was immersed in an ice-water bath. To this mixture, the above prepared THF solution of the 5,6-isopropylidine ascorbic acid was added dropwise under stirring at 0° C. over a period of 15 minutes. After the addition, the mixture was stirred at 0° C. for 40 min and at room temperature for 17 hours. The white solid of pyridinium chloride was filtered out and washed with THF (5 ml). The filtrate was concentrated to remove THF and excess pyridine to give a residue (3, FIG. 5, 2.76 g).

The crude of compound 3 (FIG. 5) was dissolved in a mixture of THF (30 ml) and 1N HCl (30 ml). The mixture was kept stirring at room temperature for 3.5 hours (TLC monitoring). The second portion of 1N HCl (10 ml) were added. The mixture was stirred for an additional 18.5 hours. The THF in the reaction mixture was removed under a reduced pressure. The water suspension was extracted with ethyl acetate and n-butanol (1:1, 110 ml). The organic layer was washed with distilled water (11 ml). The organic layer was concentrated on a rotary evaporator to give a residue. This residue was washed with hexanes (2×10 ml) and dried under the reduced pressure to give a crude product (4, FIG. 5, 1.15 g).

Preparation of sodium salt of compound 4 (FIG. 5) was similar to Example 2.

### Example 6

### Preparation of Compounds of Formulae I-III

### Synthesis of Tetrasodium Diascorbyl Diphosphate Ester of Androst-5-ene-3β,17β-diol

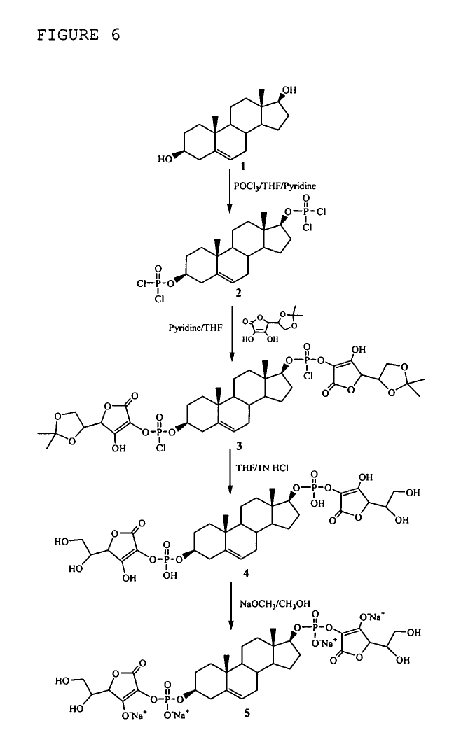

In a dry round bottom flask, androst-5-ene-3β,17β-diol (1, FIG. 6, 1.5 g, 5.17 mmol) was dissolved in pyridine (3.0 ml) and THF (15 ml). Into another dry round bottom flask was added THF (20 ml) and POCl₃ (1.17 ml, 12.56 mmol). The latter was stirirred at −5° C. for 5 minutes before the addition of androst-5-ene-3β,17β-diol (1, FIG. 6) over a period of 20 minutes. White precipitate was observed shortly after the addition of 1 (FIG. 6), and after the initial 20 minutes of reaction at −5° C., the reaction was allowed to continue at room temperature for 2.5 hours.

The flask was then cooled to 0° C., and a solution of 5,6-isopropylidene ascorbic acid (3.19 g, 14.78 mmol) in pyridine (3 ml) and THF (15 ml) was added drop-wise over a period of 20 minutes under vigorous stirring. The reaction was allowed to continue for another two hours. Then, the reaction mixture was filtered, and the filtrate was concentrated to a thick syrup. Heptane was added and the mixture was distilled under a reduced pressure. A solid crude 3 (FIG. 6) was obtained.

The crude 3 (FIG. 6) was dissolved in THF/1N HCl (1:1, 150 ml), and the hydrolysis was carried out at room temperature under vigorous stirring. After 12 hours of reaction, a TLC test indicated that the hydrolysis was complete. The THF in the reaction mixture was removed under a reduced pressure at room temperature, and n-butanol and ethyl acetate (1:1, 100 ml) was used for the extraction. The organic layer was washed with water (2×20 ml), and then concentrated to afford the crude product of diascorbyl diphosphate ester of androst-5-ene-3β,17β-diol (4, FIG. 6, 3.0 g).

The crude diascorbyl diphosphate ester of androst-5-ene-3β,17β-diol (4, FIG. 6, 400 mg) was dissolved in methanol (5 ml). To this solution was added 2 ml of sodium methoxide in methanol (20%, w/v) under magnetic stirring. White precipitate was observed upon the addition of sodium methoxide methanol solution. The suspension was stirred for half an hour before it was filtered and washed with methanol and acetone. The solid product was dried under high vacuum, and tetrasodium diascorbyl diphosphate ester of androst-5-ene-3β,17β-diol (5, FIG. 6, 330 mg) was obtained.

### Example 7

### Preparation of Compounds of Formulae IV

### Protection of Ascorbic Acid

Oleum (24%, 8.3 g) was added dropwise to acetone (50 ml). Ascorbic acid (12 g) was introduced to the mixture at 0° C. and the reaction mixture was stirred at 0° C. for 6 hours. The obtained crystals were filtered off under suction, the filtered cake was pressed to dryness and then washed with acetone (30 ml). The product, 5,6-isopropylidene ascorbic acid (14 g) was obtained.

### Example 8

### Preparation of Compounds of Formulae IV

### Attachment to Phytostanols

A solution of phytostanol mixture (24 g) (campestanol: 36.4%; sitostanol: 62.3%) in toluene (500 ml) and pyridine (25 ml) was added dropwise to a mixture of phosphorous oxychloride (9 ml) in toluene (200 ml) at 0° C. The mixture was stirred at room temperature for 3 hours. The pyridine hydrochloride was filtered off and the mother liquor was concentrated to recover the toluene. The residue was dissolved in dry THF (100 ml) and a solution of the above prepared protected ascorbic acid (14 g) in dry THF (400 ml) was added dropwise at 0° C. The stirring at room temperature was maintained for 1 hr. The solution was concentrated to remove the solvent. Ethanol (400 ml) and 3N HCl (200 ml) were added, the mixture was heated to 50° C. for 30 min and concentrated. Ethyl acetate (600 ml) was added, the resultant solution was washed with water (3×300 ml), dried over sodium sulfate, concentrated and the product (phytostanol-phosphate-ascorbate) was obtained as a white powder 22 g.

### Example 9

### Preparation of Compounds of Formulae IV

### Conversion to Sodium Salt

The above prepared acid (17 g) was dissolved in ethanol (100 ml) and a solution of sodium methoxide (2.7 g) in ethanol (50 ml) was added at stirring and at room temperature. The stirring was maintained for 30 min. after the addition. The resultant white cake was filtered off, dried and weighed, to afford a white powder 20 g (phytostanol-phosphate-ascorbate sodium).

### Example 10

### Preparation of a Composition in Accordance with the Present Invention Policosanol/FM-VP4 (DASP and DACP blend) Composition Formulation

This composition may be either compressed into tablets or filled into two-piece hard shell capsules. The formulation may be processed using both dry blending and granulation techniques.

| | Amount Per Dosage | |
|---|---|---|
| Component | Unit (mg) | Function |
| Policosanol Mixture from | 9.0-11.0 | Active agent |
| Cuban Sugar Cane, | | |
| comprising octacosanol | | |
| (C₂₈, approximately 62.9%), | | |
| triacontanol | | |
| (C₃₀, approximately 12.6%), | | |
| hexacosanol | | |
| (C₂₆, approximately 6.2%) | | |
| and other C₂₂-C₃₄ alcohols | | |
| in more minor quantities | | |
| FM-VP4 (DASP-DACP blend) | 360.0-440.0 | Active agent |
| Povidone | 20.0-40.0 | Binder |
| Cellulose | 300.0-600.0 | Binder, Diluent |
| Silicone Dioxide | 2.50-10.0 | Glidant |
| Croscarmellose Sodium | 30.0-70.0 | Disintegrant |
| Sodium Stearyl Fumarate | 5.0-20.0 | Lubricant |

### Example 11

### Policosanol/FM-VP12 (Disodium Ascorbyl Phosphate Ester of 5α-Androstan-3β-ol-17-one) Composition Formulation

This composition may be either compressed into tablets or filled into two-piece hard shell capsules. The formulation may be processed using both dry blending and granulation techniques.

| | Amount Per Dosage | |
|---|---|---|
| Component | Unit (mg) | Function |
| Policosanol- description | 9.0-11.0 | Active agent |
| as for Example 10 | | |
| FM-VP12 | 360.0-440.0 | Active agent |
| Povidone | 20.0-40.0 | Binder |
| Cellulose | 300.0-600.0 | Binder, Diluent |
| Silicone Dioxide | 2.50-10.0 | Glidant |
| Croscarmellose Sodium | 30.0-70.0 | Disintegrant |
| Sodium Stearyl Fumarate | 5.0-20.0 | Lubricant |

### Example 12

### Policosanol/FM-VP4 Composition Formulation

This composition may be filled into two-piece hard shell capsules or one-piece soft shell capsules, as a liquid dispersion fill that may be liquid, semi-solid or solid at ambient temperature (15-30 C).

| | Amount Per Dosage | |
|---|---|---|
| Component | Unit (mg) | Function |
| Policosanol- description | 9.0-11.0 | Active agent |
| as Example 10 | | |
| FM-VP4 | 360.0-440.0 | Active agent |
| Polyethylene Glycol | 50.0-490.0 | Diluent, |
| 400 | | Suspension matrix |
| Polyethylene Glycol | 100.0-490.0 | Diluent, |
| 6000-8000 | | Suspension matrix |

### Example 13

### Policosanol/FM-VP12 Composition

This composition may be filled into two-piece hard shell capsules or one-piece soft shell capsules, as a liquid dispersion fill that may be liquid, semi-solid or solid at ambient temperature (15-30 C).

| | Amount Per Dosage | |
|---|---|---|
| Component | Unit (mg) | Function |
| Policosanol- description | 9.0-11.0 | Active agent |
| as Example 10 | | |
| FM-VP12 | 360.0-440.0 | Active agent |
| Polyethylene Glycol | 50.0-490.0 | Diluent, |
| 400 | | Suspension matrix |
| Polyethylene Glycol | 50.0-490.0 | Diluent, |
| 6000-8000 | | Suspension matrix |

### REFERENCES

1. Law M. R., Wald N. J., Wu., Hacksaw Z A., Bailey A.; Systemic underestimation of association between serum cholesterol concentration and ischemic heart disease in observational studies: Data from BUPA Study; Br. Med. J. 1994; 308:363-366
2. Law M. R., Wald N. J., Thompson S. G.; By how much and how quickly does reduction in serum cholesterol concentration lower risk of ischemic heart disease? Br. Med. J. 1994; 308:367-373
3. Kannel W B, Castelli W P, Gordon T et al. Lipoprotein cholesterol in the prediction of atherosclerotic disease: new perspectives based on the Framingham Heart Study. Ann Intern Med. 1995;90:85-91
4. Singh B K, Mehta J L. Management of dyslipidemia in the primary prevention of coronary heart disease. Curr Opin Cardiol. 2002; 17:503-11
5. La Rosa J. C., Hunninghake D. Bush D. et al.; The cholesterol facts: A summary of the evidence relating to dietary fats, serum cholesterol and coronary heart disease:Ajoint statement by the American Heart Association and the National Heart, Lung and Blood Institute. Circulation 1990; 81:1721-1733
6. Havel R. J., Rapaport E. Drug Therapy: Management of Primary Hyperlipidemia. New England Journal of Medicine, 1995; 332:1491-1498
7a. DiMuro S., Exercise intolerance and the mitochondrial respiratory chain. Ital J Neurol Sci. December 1999;20(6):387-393).
7b. Carr K. Cuban Biotechnology Trends Nature 1999; 398:A22-23
8. Uribarri E., Laguan A., Sierra R., Ricardo Y. Physico-mechanical characterization of policosanol, a novel hypocholesterolemic drug. Drug Devel. Ind. Pharm. 2002:28:89-93
9. Janikula M. Policosanol: A new treatment for cardiovascular Disease? Altern Med Rev 2002; &:203-217
10. Arruzazabala ML, Carbajal D, Mas R Garcia M Fraga V. Effects of Policosanol on Platelet aggregation in rats. Thromb Res. 1993; 69:321-7
11. Moncada S, Vane J P Pharmacology and endogenous role of prostaglandin thromboxane A2 and prostacyclin. Pharmacological Reviews. 1979; 30:293-331
12. Noa M, Mas R, Mesa R. Effect of policosanol on circulating endothelial levels in experimental models in Sprague-Dawley rats and in rabbits. J. Pharm. Pharmacol. 1997; 49:999-1002
13. Noa M, de a Rosa M C, Mas R. Effect of policosanol on foam cell formation in carrageenan-induced granulomas in rats. J. Pharm Pharmacol. 1996;48:306
14. Arruzazabala M L, Molina V, Carbajal D, Valdes S, Mas Effect of Policosanol on cerebral ischemia in Mongolian gerbils: role of prostacylcin and thromboxane A2. Prostaglandin Leukot Essent Fatty Acids. 1993;49:695-7
15. Valdes S, Arruzazabala ML, Fernandez L, Mas R, Carbajal D, Aleman C, Molina V. Effect of policosanol on platelet aggregation in healthy volunteers. Pharmacol Res 1996; 16:67-72
16. Arruzazabala M L, Valdes S, Mas R, Fernandez L, Carbajal D. Effect of policosanol successive dose increases on platelet aggregation in healthy volunteers. Pharmacol Res 1996; 34:181-185
17. Carbajal D, Arruzazabala ML, Valdes S, Mas R. Effect of policosanol on platelet aggregation and serum levels of arachidonic metabolites in healthy volunteers. Prostaglandin Leukot Essent Fatty Acids. 1998;58:61-64
18. Arruzazabala M L, Valdes S, Mas R, Carbajal D, Fernandez L. Comparative study of policosanol, aspirin and the combination therapy policosanol-aspirin on platelet aggregation in healthy volunteers. Pharmacol Res 1997; 36:293-7
19. Arruzazabala M L, Mas R, Molina V, Carbajal D, Mendoza S, Fernandez L, Valdez S. Effect of policosanol on platelet aggregation in type II hypercholesterolemic patients. Int. J. Tissue React. 1998; 20:119-24
20. Menendez R, Fernandez S I, Del R10 A, Gonzalez R M, Fragas V, Amor A M, Mas R M. Policosanol inhibits cholesterol biosynthesis and enhances low density lipoprotein processing in cultured human fibroblasts. Biol. Res. 1994; 27:199-203.
21. Kuccodkar et al.; Effects of plant sterols on cholesterol metabolism. Atherosclerosis, 1976; 23:239-248
22. Lees R. S., Lees A. M. Effects of sitosterol therapy on plasma lipid and lipoprotein concentrations. In: Greten H (Ed) Lipoprotein Metabolism. Springer-Verlag, Berlin, Heidelberg, New York, 1976:119-124
23. Lees A. M., Mok H. Y. I., Lees R. S., McCluskey M. A., Grundy S. M. Plant sterols as cholesterol-lowering agents: clinical trials in patients with hypercholesterolemia and studies of sterol balance. Atherosclerosis 1977; 28: 325-338
24. Anti-hypercholesterolemic effect of dehydroepiandrosterone in rats, Proc. Soc. Expt. Biol. Med., 125, 1136-1140 (1967)
25. New Eng. J. Med. 1999; 336:973-9
26. J Path. 1997; 181: 93-99
27. Gamez R. et al. Current Therapeutic Research, Vol. 61. No. 7, July 2000
28. Clin. Drug Invest. 2003; 23(12):789-802

1. A composition comprising one or more long chain alcohols (policosanols) and/or their respective acids (policosanoic acids) and one or more ascorbic acid derivatives, including all biologically acceptable salts or solvates or prodrugs of at least one such derivative or of the salts or of the solvates thereof represented by the following general formulae: 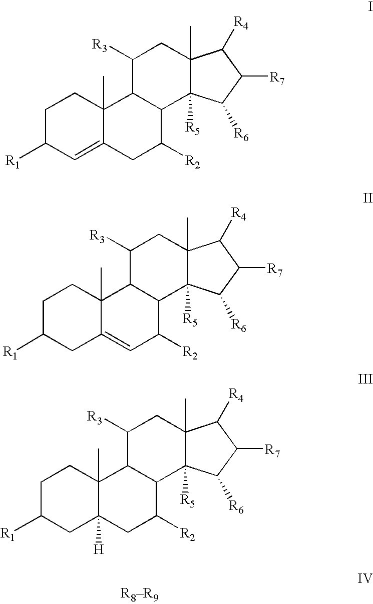
wherein, in formulae I, II and III, representing derivatives of androstene and androstane, R₁, R₂, R₃, R₄, R₅, R₆ may individually be chosen from hydrogen, OH, carbonyl, and an ascorbyl-based moiety, and wherein at least one substituent is the ascorbyl-based moiety; and R₇ may be hydrogen or any halogen; and wherein, in formula IV, R8 is an ascorbyl-based moiety and R9 is a sterol or stanol moiety.

2. The composition of claim 1, wherein, in formula IV, R₉ is a phytosterol or phytostanol selected from the group consisting of sitosterol, campesterol, stigmasterol, brassicasterol (including dihydrobrassicasterol), desmosterol, chalinosterol, poriferasterol, clionasterol, ergosterol, coprosterol, codisterol, isofucosterol, fucosterol, clerosterol, nervisterol, lathosterol, stellasterol, spinasterol, chondrillasterol, peposterol, avenasterol, isoavenasterol, fecosterol, pollinastasterol, sitostanol, campestanol, stigmastanol, brassicastanol (including dihydrobrassicastanol), desmostanol, chalinostanol, poriferastanol, clionastanol, ergostanol, coprostanol, codistanol, isofucostanol, fucostanol, clerostanol, nervistanol, lathostanol, stellastanol, spinastanol, chondrillastanol, pepostanol, avenastanol, isoavenastanol, fecostanol, and pollinastastanol.

3. The composition of claim 1 wherein, in formula IV, the sterol and stanol are in either a natural or synthesized form.

4. The composition of claim 1 wherein, in formula IV, the sterol and stanol are in any one of their isomeric forms.

5. The composition of claim 1, wherein, in formula IV, the stanol is sitostanol.

6. The composition of claim 1 comprising a disodium ascorbyl stanyl phosphate composition which comprises a blend of disodium ascorbyl campestanyl phosphate and disodium ascorbyl sitostanyl phosphate each in accordance with formula IV.

7. The composition of claim 1 wherein the policosanols are derived from sugar cane.

8. The composition of claim 1 wherein the policosanols are derived from Cuban sugar cane (Saccharum officinarum).

9. The composition of claim 1 wherein the policosanols are derived from one of rice bran wax or beeswax.

10. The composition of claim 1 wherein the policosanols are long chain alcohols comprising from 20 to 39 carbons.

11. The composition of claim 1 wherein, in each formulae, the ascorbyl-based moiety is: 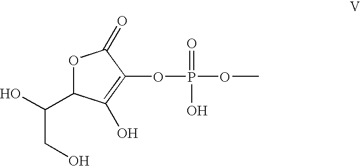
or one of its' biologically acceptable salts.

12. The composition of claim 1 wherein, in each formulae, the ascorbyl-based moiety is: 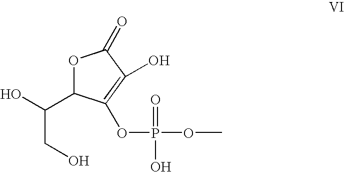
or one of its' biologically acceptable salts.

13. The composition of claim 1 wherein, each formulae, the ascorbyl-based moiety is: 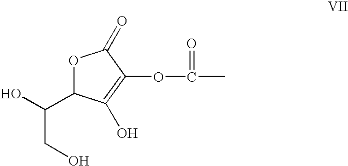
or one of its' biologically acceptable salts.

14. The composition of claim 1 wherein, in each formulae, the ascorbyl-based moiety is: 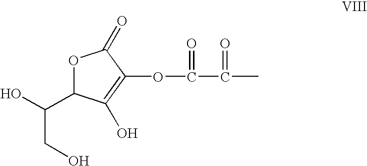
or one of its' biologically acceptable salts.

15. The composition of claim 1 wherein, in each formulae, the ascorbyl-based moiety is: 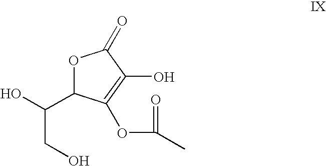
or one of its' biologically acceptable salts.

16. The composition of claim 1 wherein, in each formulae, the ascorbyl-based moiety is: 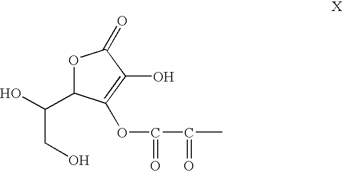
or one of its' biologically acceptable salts.

17. The composition of claim 1 wherein, in each formulae, the ascorbyl-based moiety is: 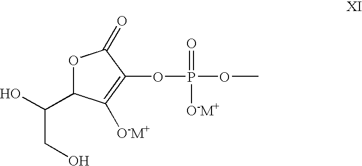
wherein M+ is selected, independently, from the group consisting of hydrogen, metals, alkali earth metals, or alkali metals.

18. The composition of claim 1 wherein, in each formulae, the ascorbyl-based moiety is: 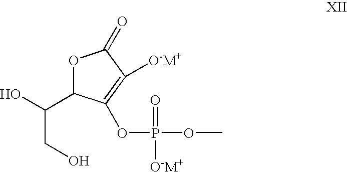
wherein M+ is selected, independently, from the group consisting of hydrogen, metals, alkali earth metals, or alkali metals.

19. The composition of claim 1 wherein, in each formulae, the ascorbyl-based moiety is: 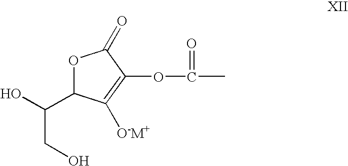
wherein M+ is selected, from the group consisting of hydrogen, metals, alkali earth metals, or alkali metals.

20. The composition of claim 1 wherein, in each formulae, the ascorbyl-based moiety is: 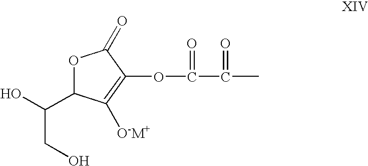
wherein M+ is selected, from the group consisting of hydrogen, metals, alkali earth metals, or alkali metals.

21. The composition of claim 1 wherein, in each formulae, the ascorbyl-based moiety is: 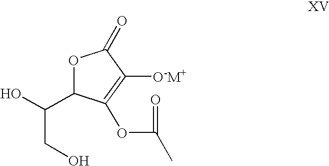
wherein M+ is selected, from the group consisting of hydrogen, metals, alkali earth metals, or alkali metals.

22. The composition of claim 1 wherein, in each formulae, the ascorbyl-based moiety is: 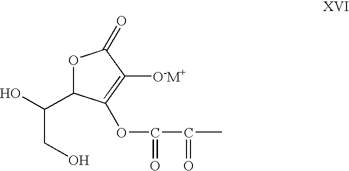
wherein M+ is selected, from the group consisting of hydrogen, metals, alkali earth metals, or alkali metals.

23. The composition of any of claims 17 to 23, wherein M+ is selected from the group consisting of calcium, magnesium, manganese, copper, zinc, sodium, potassium and lithium.

24. The composition of claim 1 wherein, in formulae I, II and III, R1 is an ascorbyl moiety, R2, R3, R5, R6 and R7 are H, and R4 is carbonyl.

18. The composition of claim 1 wherein, in formulae I, II and III, R1 is an ascorbyl moiety, R2, R3, R5 R6 and R7 are H, and R4 is OH.

19. The composition of claim 1 wherein, in formulae I, II and III and III R4 is an ascorbyl moiety, R1 is OH, and R2, R3, R5, R6 and R7 are H.

20. The composition of claim 1 wherein, in formulae I, II and III and III R4 is an ascorbyl moiety, R1 is carbonyl, and R2, R3, R5, R6 and R7 are H.

21. The composition of claim 1 wherein, in formulae I, II and III R1 and R4 are ascorbyl moieties, and R2, R3, R5, R6, and R7 are H.

22. The composition of claim 1 wherein, in formulae I, II and III and III R1 and R2 are ascorbyl moieties, R3, R5, R6 and R7 are H, and R4 is OH.

23. The composition of claim 1 wherein, in formulae I, II and III and III R1 and R2 are ascorbyl moieties, R3, R5, R6, and R7 are H, and R4 is carbonyl.

24. The composition of claim 1 wherein, in formulae I, II and III R1 and R4 are ascorbyl moieties, R2 is OH, and R3, R5, R6 and R7 are H.

25. The composition of claim 1 wherein, in formulae I, II and III and III R3 is an ascorbyl moiety, R1 and R4 are carbonyl, and R2, R5, R6 and R7 are H.

26. The composition of claim 1 wherein, in formulae I, II and III and III R3 is an ascorbyl moiety, R1 and R4 are OH, and R2, R5, R6 and R7 are H.

27. The composition of claim 1 wherein, in formulae I, II and III R5 is an ascorbyl moiety, R1 and R4 are carbonyl, and R2, R3, R6 and R7 are H.

28. The composition of claim 1 wherein, in formulae I, II and III R5 is an ascorbyl moiety, R1 and R4 are OH, and R2, R3, R6 and R7 are H.

29. The composition of claim 1 wherein, in formulae I, II and III R6 is an ascorbyl moiety, R1 and R4 are carbonyl, and R2, R3, R5 and R7 are H.

30. The composition of claim 1 wherein, in formulae I, II and III R6 is an ascorbyl moiety, R1 and R4 are OH, and R2, R3, R5 and R7 are H.

31. The composition of claim 1 wherein, in formulae I, II and III wherein R4 is an ascorbyl moiety, R1 and R2 are OH, and R3, R5, R6 and R7 are H.

32. The composition of claim 1 wherein, in formulae I, II and III R4 is an ascorbyl moiety, R1 and R3 are OH, and R2, R5, R6 and R7 are H.

33. The composition of claim 1 wherein, in formulae I, II and III R4 is an ascorbyl moiety, R1 and R3 are carbonyl, and R2, R5, R6 and R7 are H.

34. The composition of claim 1 wherein, in formulae I, II and III R4 is an ascorbyl moiety, R1 is OH, R3 is carbonyl and R2, R5, R6 and R7 are H.

35. The composition of claim 1 wherein, in formulae I, II and III R4 is an ascorbyl moiety, R1 is carbonyl, R3 is OH, and R2, R5, R6 and R7 are H.

36. The composition of claim 1 wherein, in formulae I, II and III R1 is an ascorbyl moiety, R3 and R4 are OH, and R2, R5, R6 and R7 are H.

37. The composition of claim 1 wherein, in formulae I, II and III R1 is an ascorbyl moiety, R3 and R4 are carbonyl, and R2, R5, R6 and R7 are H.

38. The composition of claim 1 wherein, in formulae I, II and III R1 is an ascorbyl moiety, R3 is OH, R4 is carbonyl, and R2, R5, R6 and R7 are H.

39. The composition of claim 1 wherein, in formulae I, II and III R1 is an ascorbyl moiety, R3 is carbonyl, R4 is OH, and R2, R5, R6 and R7 are H.

40. The composition of claim 1 wherein, in formulae I, II and III R1 is an ascorbyl moiety, R2 and R4 are OH, and R3, R5, R6 and R7 are H.

41. The composition of claim 1 wherein, in formulae I, II and III R1, R2 and R4 are ascorbyl moieties, and R3, R5, R6 and R7 are H.

42. The composition of claim 1 wherein, in formulae I, II and III R1 and R2 are ascorbyl moieties, R4 is carbonyl, and R3, R5, R6 and R7 are H.

43. The composition of claim 1 wherein, in formulae I, II and III R1 is an ascorbyl moiety, R4 is carbonyl, R2, R3, R5, R6 are H, and R7 is a halogen.

44. The composition of claim 1 wherein, in formulae I, II and III R1 and R4 are ascorbyl moieties, R2, R3, R5, R6 are H, and R7 is a halogen.

45. The composition of claim 1 wherein, in formulae I, II and III R4 is an ascorbyl moiety, R1 is carbonyl, R2, R3, R5, R6 are H, and R7 is a halogen.

46. The composition of claim 1 wherein, in formulae I, II and III R3 is an ascorbyl moiety, R4 is carbonyl, R1 is OH, R2, R5, R6 are H, and R7 is a halogen.

41. The composition of claim 1 wherein, in formulae I, II and III R3 is an ascorbyl moiety, R4 is OH, R1 is carbonyl, R2, R5, R6 are H, and R7 is a halogen.

42. The composition of claim 1 wherein, in formulae I, II and III R5 is an ascorbyl moiety, R1 and R4 are carbonyl, R2, R3, R6 are H, and R7 is a halogen.

43. The composition of claim 1 wherein, in formulae I, II and III R5 is an ascorbyl moiety, R1 and R4 are OH, R2, R3, R6 are H, and R7 is a halogen.

44. The composition of claim 1 wherein, in formulae I, II and III R6 is an ascorbyl moiety, R1 and R4 are carbonyl, R2, R3, R5 are H, and R7 is a halogen.

45. The composition of claim 1 wherein, in formulae I, II and III R6 is an ascorbyl moiety, R1 and R4 are OH, R2, R3, R5 are H, and R7 is a halogen.

46. The composition of claim 1 wherein, in formulae I, II and III R1, R3 and R4 are ascorbyl moieties, R2 and R5, R6 are H, and R7 is a halogen.

47. The composition of claim 1 wherein, in formulae I, II and III R1, R4 and R5 are ascorbyl moieties, R2 and R3, R6 are H, and R7 is a halogen.

48. The composition of claim 1 wherein, in formulae I, II and III R1, R2 and R4 are ascorbyl moieties, R3 and R5, R6 are H, and R7 is a halogen.

49. The composition of claim 1 wherein, in formulae I, II and III R1, R4, R6 are ascorbyl moieties; R2, R3, and R5 are H; and R7 is a halogen.

50. The composition of claim 1 additionally comprising one or more pharmaceutically acceptable carriers, adjuvants, excipients, binders, stabilizers, colouring agents, or stabilizers.

51. The composition of claim 1 adapted into a pharmaceutical dosage form.

50. A method of lowering serum LDL cholesterol in an animal which comprises administering a therapeutically effective amount of a composition comprising one or more long chain alcohols (policosanols) and/or their respective acids (policosanoic acids) and one or more ascorbic acid derivatives, including salts thereof, represented by the following general formulae: 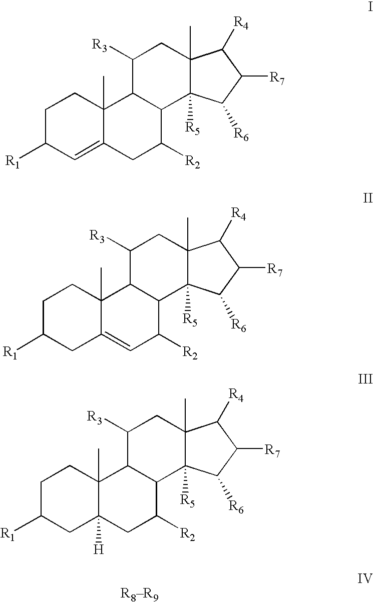
wherein, in formulae I, II and III, representing derivatives of androstene and androstane, R₁, R₂, R₃, R₄, R₅, R₆ may individually be chosen from hydrogen, OH, carbonyl, and an ascorbyl-based moiety, and wherein at least one substituent is the ascorbyl-based moiety; and R₇ may be hydrogen or any halogen; and wherein, in formula IV, R8 is an ascorbyl-based moiety and R9 is a sterol or stanol moiety.

47. A method of reducing inflammation in an animal in need of such reduction which comprises administering a therapeutically effective amount of a composition comprising one or more long chain alcohols (policosanols) and/or their respective acids (policosanoic acids) and one or more ascorbic acid derivatives, including salts thereof, represented by the following general formulae: 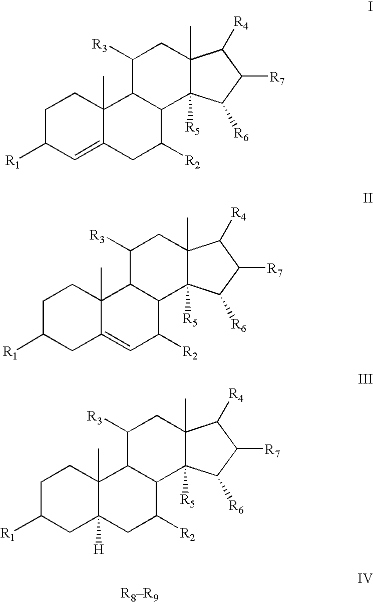
wherein, in formulae I, II and III, representing derivatives of androstene and androstane, R₁, R₂, R₃, R₄, R₅, R₆ may individually be chosen from hydrogen, OH, carbonyl, and an ascorbyl-based moiety, and wherein at least one substituent is the ascorbyl-based moiety; and R₇ may be hydrogen or any halogen; and wherein, in formula IV, R8 is an ascorbyl-based moiety and R9 is a sterol or stanol moiety.

48. A method of modulating serum lipids and triglycerides, specifically by increasing serum HDL cholesterol and/or decreasing serum triglycerides in an animal in need of such modulation, which comprises administering a therapeutically effective amount of a composition comprising one or more long chain alcohols (policosanols) and/or their respective acids (policosanoic acids) and one or more ascorbic acid derivatives, including salts thereof, represented by the following general formulae: 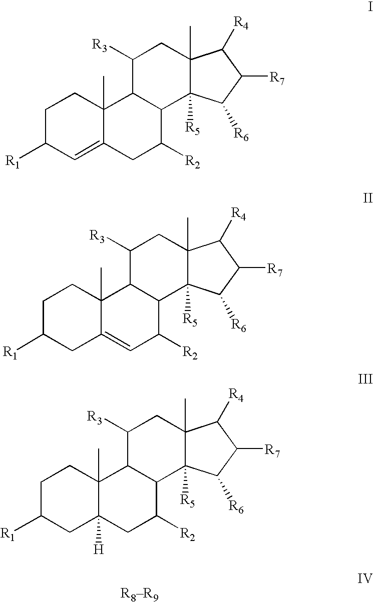
wherein, in formulae I, II and III, representing derivatives of androstene and androstane, R₁, R₂, R₃, R₄, R₅, R₆ may individually be chosen from hydrogen, OH, carbonyl, and an ascorbyl-based moiety, and wherein at least one substituent is the ascorbyl-based moiety; and R₇ may be hydrogen or any halogen; and wherein, in formula IV, R8 is an ascorbyl-based moiety and R9 is a sterol or stanol moiety.

49. The composition of claim 1 comprising from 10% to 80% by weight of component A and from 1% to 70% by weight of component B.

50. The composition of claim 1 comprising from 20% to 70% by weight of component A and from 10% to 40% by weight of component B.

51. The composition of claim 1 wherein the ratio, by weight, of component A to component B is from 1:1 to 50:1.

52. The composition of claim 1 wherein the ratio, by weight, of component A to component B [one or more ascorbic acid derivatives] is from 2:1 to 20:1.

53. The composition of claim 1 wherein, in the policosanol component, greater than 90% by weight of alcohols are C₂₀ or longer.

54. The composition of claim 1 wherein the policosanol component comprises greater than 50% by weight of octacosanol(C₂₈).

55. The composition of claim 1 wherein, in the policosanoic acid component, greater than 90% by weight of acids are C₂₀ or longer.

56. The composition of claim 1 wherein the policosanoic acid component comprises greater than 50% by weight of octacosanoic acid(C₂₈).

57. The composition of claim 1 wherein the policosanoic acid component comprises greater than 60% by weight of octacosanoic acid(C₂₈).

58. The composition of claim 1 comprising at least 60% by weight octacosanol, from 9-15% triacontanol and from 4-10% hexacosanol.
